SEQUENCE LISTING (14 sequences)

SEQ ID NO: 1 - DNA, 23 bp, synthetic construct
  source 1..23: mol_type = other DNA
aagatcgccgtgtaagaattccg

SEQ ID NO: 2 - DNA, 22 bp, synthetic construct
  source 1..22: mol_type = other DNA
tgcccgcggttatcatcgtgtt

SEQ ID NO: 3 - DNA, 23 bp, synthetic construct
  source 1..23: mol_type = other DNA
tgcatagacggtgctttacttac

SEQ ID NO: 4 - DNA, 23 bp, synthetic construct
  source 1..23: mol_type = other DNA
ggtacaagatcaattggttgctc

SEQ ID NO: 5 - DNA, 22 bp, synthetic construct
  source 1..22: mol_type = other DNA
ggtggcaaaccttgtatcaaag

SEQ ID NO: 6 - DNA, 23 bp, synthetic construct
  source 1..23: mol_type = other DNA
gaggctatagcttgtaaggttgc

SEQ ID NO: 7 - DNA, 21 bp, synthetic construct
  source 1..21: mol_type = other DNA
cagggctcagaatatgactat

SEQ ID NO: 8 - DNA, 21 bp, synthetic construct
  source 1..21: mol_type = other DNA
gtgtaggtgcctgtgtaggat

SEQ ID NO: 9 - DNA, 22 bp, synthetic construct
  source 1..22: mol_type = other DNA
ctttggggtactgctgttatgt

SEQ ID NO: 10 - DNA, 22 bp, synthetic construct
  source 1..22: mol_type = other DNA
catctccttcaccttcaccaga

SEQ ID NO: 11 - DNA, 38 bp, synthetic construct
  source 1..38: mol_type = other DNA
atctcttgtagatctgttctctaaacgaacaaactaaa

SEQ ID NO: 12 - DNA, 20 bp, synthetic construct
  source 1..20: mol_type = other DNA
atgtctgataatggacccca

SEQ ID NO: 13 - DNA, 20 bp, synthetic construct
  source 1..20: mol_type = other DNA
ttaggcctgagttgagtcag

SEQ ID NO: 14 - DNA, 27952 bp, synthetic construct
  source 1..27952: mol_type = other DNA
acctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatttgagcctgggagctctctggctaactagggaacccactgcttaagcctcaataggccggcctaatacgactcactataggggggagaggcccgaaggccctgatgagtccgtgaggacgaaacggtacccggtaccgtcattaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggagggccgcggggaagcggagctactaacttcagcctgctgaagcaggctggagacgtggaggagaaccctggacctatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaagaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataaccgcgggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaagcggccgcacgaacaatggtttctaagggagaagaactctttactggtgttgtcccaattctggttgagctggatggtgatgtgaatggccacaaattctctgtgtctggtgaaggtgaaggagatgcaacttatggaaagctgactctgaagttcatttgtacaacaggaaagctgccagtgccttggccaactctggtgaccaccctgacttatggtgttcaatgtttcagcaggtaccctgaccacatgaagcagcatgacttctttaaatctgcaatgccagaaggttatgttcaggagaggacaatcttctttaaggatgatggaaattataagacaagggcagaagtgaagtttgaaggtgatacactggttaacagaattgagctgaaaggcattgattttaaggaagatggaaacattctgggtcacaagctggagtacaactataattctcacaatgtttacattatggcagataagcagaggaatggaattaaggctaatttcaagattagacacaacattgaggatggatctgtccaactggcagaccattaccagcagaacacccctattggtgatggcccagttctcctcccagataatcactatctcagcactcaatctgctctgtccaaagaccctaatgagaaaagagaccacatggtcctcctggagtttgtgacagcagcaggaattactctgggaatggatgagctgtacaagggtaagtcactgactgtctatgcctgggaaagggtgggcaggagatggggcagtgcaggaaaagtggcactatgaacccactagtttgacaattaatcataagcatagtataatacaactcactatagcaattgtactaaccttcttctctttcctctcctgacaggaggagccatcatgaagaccttcaacatctctcagcaggaccttgagctggtggaagttgccactgagaaaatcaccatgctctatgaggacaacaagcaccatgttggtgctgccatcaggaccaagacaggagaaatcatctctgctgtccacattgaagcctacattggcagggtcactgtgtgtgcagaggccattgccattgggtctgcagtctccaatgggcagaaggactttgacaccattgtggctgtgaggcacccctacagtgatgaggtggacaggtccatcagagtggtgtccccctgtggcatgtgcagggagctcatctcagactatgcccctgattgctttgttctgattgagatgaatggcaagctggtcaagacaaccattgaggagctgattccactgaaatacaccagaaactaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaagggcggcatggtcccagcctcctcgctggcgccgcctgggcaacatgcttcggcatggcgaatgggaccggatccgggggcgcgcctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

(19) United States
(12) Patent Application Publication
(10) Pub. No.: US 2023/0304041 A1
(43) Pub. Date: Sep. 28, 2023

(54) DUAL PROMOTER-DRIVEN AND DUAL REPORTER-EXPRESSING SARS-COV-2 REPLICONS AND METHODS OF USE

(71) Applicant: Rosalind Franklin University of Medicine & Science, North Chicago, IL (US)

(72) Inventors: Johnny HE, North Chicago, IL (US); Ying LIU, North Chicago, IL (US)

(21) Appl. No.: 18/188,932

(22) Filed: Mar. 23, 2023

Related U.S. Application Data
(60) Provisional application No. 63/323,788, filed on Mar. 25, 2022.

(51) Int. Cl. C12N 15/86; C12Q 1/70

(52) U.S. Cl. CPC C12N 15/86; C12Q 1/701; C12Q 2600/136; C12Q 2600/158; C12N 2770/20043

(57) ABSTRACT

The present disclosure provides constructs and kits comprising SARS-CoV-2 (SARS2) replicons and methods for screening and/or evaluating anti-SARS2 drugs using the replicons.

### CROSS REFERENCE TO RELATED APPLICATIONS

This application is a non-provisional application of U.S. Provisional Application 63/323,788, filed Mar. 25, 2022, the disclosure of which is incorporated by reference in its entirety.

### REFERENCE TO SEQUENCE LISTING

A computer readable form of the Sequence Listing is filed with this application by electronic submission and is incorporated into this application by reference in its entirety. The Sequence Listing is contained in the ST.26 XML file created on Mar. 21, 2023, having the file name “20-1939-US_Sequence-Listing.xml” and is 41,216 bytes in size.

### BACKGROUND

Coronavirus disease in 2019 (COVID-19) is a highly transmissible respiratory disease and caused by SARS-CoV-2 (SARS2) [1-4]. More than 450 million people have been infected and more than 6 million have died from this infection worldwide since late 2019 [5]. Infected people develop headache, fever, coughing, diarrhea, and pneumonia with a higher mortality in the elderly and those with compromised immune system, diabetes, and other chronic respiratory and heart diseases [6, 7]. Thus, COVID-19 has been a profound global health threat. The method of choice for detection and diagnosis of SARS2 infection is real-time RT-PCR, which is very sensitive and accurate and has successfully been adapted for various human specimens including nasal swabs and sputum [8, 9]. Vaccines against SARS2 have been proven to be effective in preventing viral transmission and improving clinical outcomes [10-13]. Antiviral drugs Remdesivir and Molnupiravir can be used to treat and prevent COVID-19, but with a very modest efficacy [14-19]. In addition, over 30% of the population who were infected with SARS2 and recovered from COVID-19 have experienced the long-COVID symptoms [20-25]. The continued emergence of SARS2 variants, lack of effective and direct SARS2 targeting drugs, and a large population with long-COVID symptoms make it imperative to develop more effective and SARS2-specific antiviral drugs to treat and prevent the disease.

SARS2 belongs to a member of the coronavirus family and is a single-stranded positive enveloped RNA virus [26-28]. The viral genome is about 30 kb nucleotides in length and has a leader cap structure and an untranslated region (UTR) at the 5′ end and an UTR and a poly(A) tail at the 3′ end. Both UTR form highly specified RNA structures required for viral RNA translation, transcription, and replication. There are 14 open reading frames (ORF) within the viral genome, preceded by transcriptional regulatory sequences (TRS). The two main ORF's are ORF1a and ORF1b. ORF1a encodes a large polyprotein, which is cleaved into nonstructural proteins (NSP) 1-11. ORF1b is derived from the frameshift at the 3′ end of ORF1a and as result, encodes another large polyprotein, which is cleaved into NSP1-10 and NSP12-16. All these NSP make up the replication/transcription complex with distinct functions and are essential for viral RNA replication/transcription, which includes first synthesis of negative-stranded viral genomic RNA (gRNA) and subgenomic RNA (sgRNA) and subsequent synthesis positive-stranded gRNA and sgRNA. Four structural proteins: spike S, envelope E, membrane M, and nucleocaspid N, and nine accessory proteins: ORF3a/b, 6, 7a/b, 8, 9a/b and 10 are encoded by their corresponding sgRNA. Newly synthesized gRNA and structural proteins are assembled to form new infectious virions for the new round of infection. Because of the unique features and viral proteins (enzymes) involved, the RNA replication and transcription process affords the best targets to develop direct SARS2 targeting antiviral drugs.

Replicons have been the strategy of choice to screen and identify antiviral drugs for positive-stranded RNA viruses and to study molecular mechanisms of the viral replication process. They are constructed by reverse genetic engineering of partial viral genome and one or more structural genes so that the viral genome can replicate and persist in cells. Replicons have successfully been constructed in several families of positive-stranded RNA viruses including picornaviridae [29], caliciviridae [30], flaviviridae [31-36], and coronaviridae [37-41]. A great example is HCV replicons, which have been attributed to successful identification of several direct acting antivirals to treat HCV infections [42, 43]. All the replicons lack the envelope gene and other structural genes, and there are no infectious viruses produced from use of the replicons. Thus, replicons represent an ideal platform for identification of anti-SARS2 antiviral drugs and elucidation of molecular mechanisms of SARS2 replication for researchers, particularly for those who do not have access to a research facility of biosafety level 3 or higher required for working with SARS2.

### SUMMARY OF THE DISCLOSURE

The severe acute respiratory syndrome coronavirus-2 (SARS CoV-2, SARS2) remains a great global health threat and demands identification of more effective and SARS2-targeted antiviral drugs even with successful development of anti-SARS2 vaccines. Viral replicons have proven to be a rapid, safe and readily scalable platform for high throughput screening, identification and evaluation of antiviral drugs against positive-stranded RNA viruses.

This disclosure provides a unique robust SARS2 replicon with dual promoters HIV long terminal repeat (LTR) and T7 and with dual reporters luciferase and green fluorescent protein. Several other novel features were also incorporated into the design of the replicon, which together afford the replication fidelity of the replicon, maximized expression of the full-length replicon, and flexible monitoring of the replicon replication. These findings indicate that the replicon may provide a platform for rapid, sensitive, and safe screening and evaluation of the SARS2 replication inhibitors.

The genomic organization of the replicon was designed with features that were to ensure the replication fidelity of the replicon, to maximize the expression of the full-length replicon, and to offer the monitoring flexibility of the replicon replication. This disclosure demonstrates the success of the construction of the replicon and expression of reporter genes and SARS structural nucleocapsid N from the replicon DNA or the RNA that was in vitro transcribed from the replicon DNA. This disclosure demonstrates detection of the negative-stranded genomic RNA (gRNA) and subgenomic RNA (sgRNA) intermediates, a hallmark of replication of positive-stranded RNA viruses from the replicon. Lastly, this disclosure demonstrates expression of the reporter genes, nucleocapsid N gene, gRNA and sgRNA from the replicon was sensitive to inhibition by Remdesivir. Taken together, this disclosure supports the use of the replicon construct for identification of anti-SARS2 drugs and development of new anti-SARS strategies targeted at the step of the virus replication.

In one aspect, this disclosure provides a construct comprising a SARS-CoV-2 (SARS2) replicon, wherein the construct comprises 5′ to 3′:
a) two or more promoter sequences;
b) a nucleic acid sequence encoding SARS2 non-structural protein 1;
c) a first reporter gene;
d) a nucleic acid sequence encoding SARS2 non-structural proteins 2-16;
e) a second reporter gene; and
f) a nucleic acid sequence encoding SARS2 nucleocapsid protein.

In some embodiments, the construct further comprises a 5′ untranslated region (UTR) that is 5′ of the nucleic acid sequence encoding SARS2 non-structural protein 1. In some embodiments, the 5′ UTR is 5′ of the nucleic acid sequence encoding SARS2 non-structural protein 1, and is 3′ of the two or more promoter sequences. In some embodiments, the construct further comprises a 3′ untranslated region (UTR) that is 3′ of the nucleic acid sequence encoding SARS2 nucleocapsid protein. In some embodiments, the two or more promoters comprise a eukaryotic promoter and a prokaryotic promoter.

In some embodiments, the eukaryotic promoter is selected from a HIV-1 long terminal repeat (LTR) promoter, a HIV-2 LTR promoter, a beta actin promoter, a cytomegalovirus (CMV) promoter, an human elongation factor-1 alpha (EF-1 alpha; EF1a) promoter, a phosphoglycerate kinase (PGK1) promoter, a simian virus 40 (SV40) promoter, a polyubiquitin C gene (UBC) promoter, a human T-lymphotropic virus type 1 (HTLV-1) LTR promoter, a human T-lymphotropic virus type 2 (HTLV-2) LTR promoter, a simian immunodeficiency virus (SIV) LTR promoter, a visna virus LTR promoter, a feline immunodeficiency virus (FIV) LTR promoter, an equine infectious anemia virus (EIAV) LTR promoter, a simian T-cell lymphoma virus (STLV) LTR promoter, a bovine leukemia virus (BLV) LTR promoter, a simian foamy virus (SFV) LTR promoter, and a bovine foamy virus (BFV) LTR promoter.

In some embodiments, the prokaryotic promoter is selected from a T7 promoter, a lac promoter, a Sp6 promoter, a tac promoter, a tet promoter, a trp promoter, a trc promoter, a T3 promoter, and a T5 promoter.

In some embodiments, the first reporter gene and the second reporter gene are optically detectable. In some embodiments, the first reporter gene and the second reporter gene are optically distinguishable.

In another aspect, this disclosure provides a construct comprising a SARS-CoV-2 (SARS2) replicon, wherein the construct comprises 5′ to 3′:
a) an HIV-1 promoter sequence;
b) a T7 promoter sequence;
c) a hammerhead virus ribozyme sequence;
d) a SARS2 5′ UTR sequence;
e) a nucleic acid sequence encoding SARS2 non-structural protein 1;
f) a P2A self-cleaving peptide sequence;
g) a first reporter gene;
h) an internal ribozyme entry site;
j) a nucleic acid sequence encoding SARS2 non-structural proteins 2-16;
k) a second reporter gene;
l) a nucleic acid sequence encoding SARS2 nucleocapsid protein;
m) a 3′ UTR sequence;
n) a hepatitis delta virus ribozyme sequence; and
o) a poly-A tail sequence.

In some embodiments, the construct as disclosed herein comprises the sequence of SEQ ID NO:14.

In yet another aspect, this disclosure provides a method for screening a test compound for anti-SARS2 activity, wherein the method comprises: a) incubating the test compound with a cell line comprising the construct of claim 1; and b) assaying for the presence of expression of the first reporter gene or expression of the second reporter gene, wherein a decreased expression of the first reporter gene or a decreased expression of the second reporter gene compared to a control indicates that the test compound comprises anti-SARS2 activity.

In yet another aspect, this disclosure provides a kit for screening a test compound for anti-SARS2 activity, wherein the kit comprises the construct as disclosed herein. In some embodiments, the kit further comprises: one or more primer sets to detect one or more genes of the construct; one or more cell lines to be transfected with the construct; or reagents for detection and quantitation of the two or more reporter genes of the construct.

These and other features and advantages of the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

FIGS. 1A-1B show a scheme of SARS2 genome and its replicon DNA construct. FIG. 1A. The full-length of SARS2 genome from the Wuhan-Hu-1 isolate (GenBank accession No. NC_045512.2) encodes 5′ untranslated region (UTR), nonstructural proteins NSP1-16, structural proteins S, E, M, and N, accessory proteins ORF3-10, and 3′UTR. FIG. 1B. Several genetic elements were included in the recombinant replicon DNA construct for various purposes. These include HIV long terminal repeat (LTR) promoter, T7 promoter, hammerhead virus ribozyme (HHV Rz) at the 5′ end, porcine teschovirus-1 self-cleaving peptide 2A (P2A) between NSP1 aa1-183 and firefly lurciferase, encephalomyocarditis virus internal ribosome entry site (IRES) before NSP2-16, green fluorescence protein-blasticidine (GFP::Bsr) in place of S/E/M, and hepatitis delta virus ribozyme (HDV Rz) and bovine growth hormone polyadenylation signal (BGH pA) at the 3′ end.

FIGS. 2A-2C show the construction of the SARS2 replicon DNA. FIG. 2A. The full-length of the recombinant SARS2 replicon DNA (27,952 bp) was divided to and synthesized in 5 fragments (F1/F6 and F2-F5) in the backbone of pMX backbone vector (for F2-5) or pMK backbone vector (for F1/6) with approximate nucleotide sequences for BsaI or SalI restriction sites at both 5′ and 3′ end. FIG. 2B. Fragments F2-5 in pMX were ligated to create pMX.F2-5 construct using a Golden Gate Assembly kit. FIG. 2C. pMXF2-5 and pMKF1/6 were digested with SalI and ligated to create the full-length recombinant non-infectious SARS2 replicon DNA construct using a homology recombination-based Gibson Assembly kit.

FIGS. 3A-3D show recombinant SARS2 replicon DNA and its transcribed replicon RNA. FIG. 3A. The intermediate product pMXF2-5 DNA from the Golden Gate Assembly and pMK.F1/6 DNA were confirmed using 0.5% agarose gel electrophoresis. FIG. 3B. The SARS replicon DNA was obtained from pMX.F2-5 and pMK.F1/6 using a Golden Gate Assembly kit and confirmed using 0.5% agarose gel electrophoresis, marked by an arrow. FIG. 3C. The full-length recombinant SARS2 replicon DNA was confirmed by PCR using primer pairs spanning specific junctions between two adjacent DNA fragments. FIG. 3D. SARS2 DNA replicon was used to synthesize SARS2 RNA replicon using an in vitro T7 transcription kit, and the RNA replicon was confirmed using denatured agarose electrophoresis (0.7%), marked by an arrow. Stand's: DNA size markers.

FIGS. 4A-4C show a luciferase reporter gene expression from recombinant SARS2 replicon DNA in response to HIV Tat expression. FIG. 4A & FIG. 4B. 293T were plated at a density of 1.5×10⁵ cells/well in a 24-well plate, transfected with 0.4 μg SARS2 replicon DNA and an increasing amount of pc3.Tat, cultured for 24 hr, and harvested for the luciferase activity assay (A), or transfected with 0.4 μg SARS2 replicon DNA and 0.12 μg pc3.Tat, cultured for different lengths of time, and harvested for the luciferase activity assay (B). FIG. 4C. Vero6 were transfected 0.4 μg SARS2 replicon DNA and 0.12 μg pc3.Tat, cultured for different lengths of time, and harvested for the luciferase activity assay. pcDNA3 was used to equalize the total amount of DNA among all transfections. The data were representative of at least three independent experiments.

FIGS. 5A-5B show a luciferase reporter gene expression from SARS2 replicon RNA in response to HIV Tat expression. 293T (FIG. 5A) or Vero6 (FIG. 5B) were plated at a density of 1.5×10⁵ cells/well for 293T and 1.5×10⁵ cells/well for Vero6 in a 24-well plate, transfected with 0.3 μg SARS2 replicon RNA and 0.1 μg pc3.Tat, cultured for different lengths of time, and harvested for the luciferase activity assay. pcDNA3 was used to equalize the total amount of DNA among all transfections. The data were representative of at least three independent experiments.

FIGS. 6A-6C show expression of the GFP reporter gene and SARS2 N. FIG. 6A & FIG. 6B. 293T were plated at a density of 4×10⁶ cells/plate in a 10 cm plate, transfected with 10 μg SARS2 replicon DNA and 3.3 μg pc3.Tat, cultured for different lengths of time, and harvested for direct imaging of the GFP signal at 488 nM (A), or for Western blotting against an anti-SARS2 N antibody (B). Untx: 293T were only transfected with pcDNA3. FIG. 6C. 293T were at a density of 4×10⁶ cells/plate in a 10 cm plate, transfected with 7.5 μg SARS2 replicon RNA, cultured for different lengths of time, and harvested for Western blotting against an anti-SARS2 N antibody. Western blotting against an anti-β-actin antibody was included as the equal loading control. The data were representative of at least three independent experiments.

FIGS. 7A-7B show expression of positive/negative-stranded genomic RNA (gRNA) and N subgenomic RNA (sgRNA) from the SARS2 replicon and its response to Remdesivir. FIG. 7A. Different RT primers (N3′ and LRS-L) in combination with different PCR primers (N5′/N3′ and TSR-L/N3′) were designed to distinguish positive-stranded from negative-stranded gRNA and N sgRNA. RT with N3′, followed by PCR with N5′/N3′ and TSR-L/N3′ represented positive-stranded gRNA and N sgRNA, respectively. RT with TSR-L, followed by PCR with N5′/N3′ and TSR-L/N3′ represented negative-stranded gRNA and N sgRNA, respectively. FIG. 7B. 293T were plated at a density of 6.5×10⁵ cells/well in a 6-well plate, treated with 0, 5, or 10 μM Remdesivir for 1 hr, transfected with 1.5 μg SARS2 replicon DNA and 0.5 μg pcDNA3, 1.5 μg SARS2 DNA and 0.5 μg pc3.Tat, or 1.2 μg SARS2 replicon RNA, cultured in the presence of Remdesivir for 24 hr, and harvested for RNA isolation. RT was performed using N3′ or TRS-L5′ as the primer and 0.1 0.5 μg RNA in a 25 μl reaction. An aliquot RT reaction (2 μl from N3′ RT reaction; 2 μl from TSR-L5′ RT reaction) were used as the template for PCR with indicated primer pairs. The PCR products were analyzed on 1% agarose gel electrophoresis. RT was performed using 0.1 μg RNA. RT with oligo d(T)₂₃ as the RT primer and PCR with β-actin-specific primers were performed and included as the equal loading control. Stand's: DNA size markers. Ctrl: untransfected cells. The data were representative of at least three independent experiments.

FIGS. 8A-8C show the effects of Remdesivir on gene expression from the SARS2 replicon DNA and RNA. FIG. 8A & FIG. 8B. 293T were at a density of 4×10⁶ cells/plate in a 10 cm plate, treated with Remdesivir for 1 hr, transfected with 10 μg SARS2 replicon DNA, cultured in the presence of Remdesivir for 24 hr, and harvested for the luciferase activity assay (FIG. 8A), or for Western blotting against an anti-SARS2 N antibody or anti-β-actin antibody, or by direct imaging of the GFP signal at 488 nm (FIG. 8B). FIG. 8C. 293T were at a density of 1.5×10⁵ cells/well in a 24-well plate, treated with 10 μM Remdesivir for 1 hr, transfected with 0.4 μg SARS2 replicon DNA and 0.12 μg pcDNA3, 0.4 μg SARS2 DNA and 0.12 μg pc3.Tat, or 0.3 μg SARS2 replicon RNA and 0.1 μg yeast tRNA, cultured in the presence of Remdesivir for 24 hr, and harvested for the luciferase activity assay. The controls for Remdesivir treatment were DMSO, the solvent for Remdesivir. The data were representative of at least three independent experiments.

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present disclosure.

### DETAILED DESCRIPTION OF THE DISCLOSURE

Before describing the present disclosure in detail, a number of terms will be defined. Unless otherwise required by context, singular terms shall include pluralities and plural terms shall include the singular. For example, the singular forms “a,” “an,” and “the” include plural referents unless the context clearly dictates otherwise. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives unless otherwise indicated.

In the present disclosure, any concentration range, percentage range, ratio range, or integer range is to be understood to include the value of any integer within the recited range and, when appropriate, fractions thereof (such as one tenth and one hundredth of an integer), unless otherwise indicated.

As used herein, the term “about” means ±10% of the indicated range, value, sequence, or structure, unless otherwise indicated.

It is noted that terms like “preferably,” “commonly,” and “typically” are not utilized herein to limit the scope of the claimed subject matter or to imply that certain features are critical, essential, or even important to the structure or function of the claimed subject matter. Rather, these terms are merely intended to highlight alternative or additional features that can or cannot be utilized in a particular embodiment of the present disclosure.

For the purposes of describing and defining the present disclosure it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Unless expressly specified otherwise, the term “comprising” is used in the context of the present disclosure to indicate that further members may optionally be present in addition to the members of the list introduced by “comprising”. It is, however, contemplated as a specific embodiment of the present disclosure that the term “comprising” encompasses the possibility of no further members being present, i.e. for the purpose of this embodiment “comprising” is to be understood as having the meaning of “consisting of”.

As utilized in accordance with the present disclosure, unless otherwise indicated, all technical and scientific terms shall be understood to have the same meaning as commonly understood by one of ordinary skill in the art.

Methods well known to those skilled in the art can be used to construct genetic expression constructs and recombinant cells according to this disclosure. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, CA).

As used herein, the terms “polynucleotide,” “nucleotide,” and “nucleic acid” can be used interchangeably to refer to nucleic acid comprising DNA, RNA, derivatives thereof, or combinations thereof, in either single-stranded or double-stranded embodiments depending on context as understood by the skilled worker. In the present disclosure, a “nucleic acid” molecule can include, DNA, cDNA and genomic DNA sequences, RNA, messenger RNA, and synthetic nucleic acid sequences. In some embodiments, the nucleic acid molecules are codon-optimized for expression. Thus, “nucleic acid” also encompasses embodiments in which analogs of DNA and RNA are employed. In some embodiments, the nucleic acid component may comprises one or more RNA molecules, such as viral RNA molecules or mRNA molecules that encode the protein of interest.

Disclosed herein is a novel non-infectious SARS2 replicon system. The SARS2 replicon construct comprises the following components in the direction from 5′ end to 3′ end: an HIV LTR promoter (for in vivo DNA transfection and efficient transcription of full-length RNA), a T7 promoter (for in vitro RNA transcription), a 5′ UTR (for RNA replication and anti-innate immunity), a P2A self cleaving sequence, a first reporter gene (e.g., firefly luciferase, which can be for replication monitoring by a luciferase assay, which is easy to scale up for high throughput screening), an IRES sequence (to facilitate efficient translation of the extremely large polyprotein), a TRS for GFP:Bsr subgenomic RNA transcription, GFP:Bsr (GFP offers the second sensitive monitoring of viral replication, blasticidin (Bsr) for stable replicon selection and long-term maintenance), a TRS for N subgenomic RNA transcription, N (for viral replication), orf10 3′ UTR (for RNA replication), poly-A) (polyadenylation signal), Rz at both 5′ end and 3′ end (ribozyme self-cleavage site to remove unwanted nucleotides from replicon RNA), and BGH poly-A (bovine growth hormone polyadenylation signal).

This platform will permit versatile, fast, easy, accurate and safe identification of anti-SARS2 inhibitors targeting various virus specific enzymes and different stages of virus replication and studies of basic virology of the virus.

The SARS2 replicon as described herein provides: (1) both delivery options of in vitro transcription RNA and recombinant DNA; (2) SARS2 non-structural genes, plus only structural N gene, therefore it is non-infectious (being safe, does not require BSL-3 or higher facility); (3) the HIV LTR promoter (in combination with Tat) allows abundant synthesis of the full-length RNA of about 28,000 nucleotides; (4) three reporters embedded in the system: luciferase, GFP and blasticidin, for fast, easy, and accurate monitoring of the surrogate virus replication; (5) an ECMV IRES, which will allow efficient translation of the polyprotein of about 7,000 amino acids. (6) a 5′ UTR-NSP1 and N-orf10 3′ UTR at the both ends of the SARS2 viral components of the construct, which allows for proper configuration of the RNA secondary structures within 5′ UTR and 3′ UTR for viral replication. (7) a Hammerhead virus ribozyme site at the 5′ end and a Hepatitis virus ribozyme site at the 3′ end that would allow production of RNA replicon with both 5′ and 3′ ends that are authentic to SARS2.

In one aspect, this disclosure provides a construct comprising a SARS-CoV-2 (SARS2) replicon, wherein the construct comprises 5′ to 3′:
a) two or more promoter sequences;
b) a nucleic acid sequence encoding SARS2 non-structural protein 1;
c) a first reporter gene;
d) a nucleic acid sequence encoding SARS2 non-structural proteins 2-16;
e) a second reporter gene; and
f) a nucleic acid sequence encoding SARS2 nucleocapsid protein.

As used herein, the terms “construct”, “vector” or “expression cassette”, refer to a nucleic acid construct that, when introduced into a host cell, results in transcription and/or translation of an RNA or polypeptide, respectively.

In some embodiments, the construct further comprises a 5′ untranslated region (UTR) that is 5′ of the nucleic acid sequence encoding SARS2 non-structural protein 1. In some embodiments, the 5′ UTR is 5′ of the nucleic acid sequence encoding SARS2 non-structural protein 1, and is 3′ of the two or more promoter sequences. In some embodiments, the construct further comprises a 3′ untranslated region (UTR) that is 3′ of the nucleic acid sequence encoding SARS2 nucleocapsid protein.

As used herein, the term “promoter,” refers to a polynucleotide sequence capable of driving transcription of a DNA sequence in a cell. Thus, promoters used in the constructs as disclosed herein, can include cis- and trans-acting transcriptional control elements and regulatory sequences that are involved in regulating or modulating the timing and/or rate of transcription of a gene in the construct. Reference to a promoter by name can include a wild type, native promoter as well as variants of the promoter that retain the ability to induce expression. Reference to a promoter by name is not restricted to a particular species, but also encompasses a promoter from a corresponding gene in other species.

The particular promoter employed to control the expression of a nucleic acid sequence of interest is not believed to be important, so long as it is capable of directing the expression of the polynucleotide in the targeted cell. Thus, where a human cell is targeted the nucleic acid sequence-coding region may, for example, be placed adjacent to and under the control of a promoter that is capable of being expressed in a human cell. Generally speaking, such a promoter might include either a human or viral promoter. The use of other viral or mammalian cellular or bacterial phage promoters which are well known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. In some embodiments, the two or more promoters of the construct as disclosed herein, comprise a eukaryotic promoter and a prokaryotic promoter. In some embodiments, the eukaryotic promoter is selected from a HIV-1 long terminal repeat (LTR) promoter, a HIV-2 LTR promoter, a beta actin promoter, a cytomegalovirus (CMV) promoter, an human elongation factor-1 alpha (EF-1 alpha; EF1a) promoter, a phosphoglycerate kinase (PGK1) promoter, a simian virus 40 (SV40) promoter, a polyubiquitin C gene (UBC) promoter, a human T-lymphotropic virus type 1 (HTLV-1) LTR promoter, a human T-lymphotropic virus type 2 (HTLV-2) LTR promoter, a simian immunodeficiency virus (SIV) LTR promoter, a visna virus LTR promoter, a feline immunodeficiency virus (FIV) LTR promoter, an equine infectious anemia virus (EIAV) LTR promoter, a simian T-cell lymphoma virus (STLV) LTR promoter, a bovine leukemia virus (BLV) LTR promoter, a simian foamy virus (SFV) LTR promoter, and a bovine foamy virus (BFV) LTR promoter. In some embodiments, the eukaryotic promoter is a HIV-1 long terminal repeat (LTR) promoter. In some embodiments, the prokaryotic promoter is selected from a T7 promoter, a lac promoter, a Sp6 promoter, a tac promoter, a tet promoter, a trp promoter, a trc promoter, a T3 promoter, and a T5 promoter. In some embodiments, the prokaryotic promoter is a T7 promoter.

In some embodiments, the two or more promoters are operably linked to one or more genes in the construct. As used herein, the term “operably linked” refers to a functional relationship between two or more nucleic acid sequences of the construct. Typically, it refers to the functional relationship of a transcriptional regulatory promoter sequence to a transcribed gene sequence. For example, a promoter is operably linked to a DNA or RNA sequence if it stimulates or modulates the transcription of the DNA or RNA sequence in an appropriate host cell or other expression system. Generally, promoter transcriptional regulatory sequences that are operably linked to a transcribed gene sequence are physically contiguous to the transcribed sequence.

As used herein, a “reporter gene” refers to a nucleic acid sequence encoding a protein product that can generate, under appropriate conditions, a detectable signal that allows detection for indicating the presence and/or quantity of the reporter gene protein product. In some embodiments, the first reporter gene or the second reporter gene is a nucleic acid sequence encoding a protein that allows a cell to present a detectable signal. Examples of such a protein capable of generating a detectable signal include a protein that generates a fluorescence signal or a phosphorescence signal, a protein that is detectable in an assay, or a protein exhibiting an enzyme activity. Examples of a protein encoded by the first reporter gene or the second reporter gene can include fluorescent proteins such as a green fluorescent protein (GFP), a humanized Renilla green fluorescent protein (hrGEP), a blue fluorescent protein (BFP), a cyan fluorescent protein (CFP), a yellow fluorescent protein (YFP), a red fluorescent protein (RFP or DsRed), and enhanced or modified versions thereof. Additional examples of a protein encoded by the first reporter gene or the second reporter gene can include bioluminescent proteins such as firefly luciferase and Renilla luciferase. Further examples of a protein encoded by such a reporter gene include enzymes for converting chemiluminescent substrates, such as alkaline phosphatase, peroxidase, chloramphenicol acetyltransferase, and β-galactosidase. In some embodiments, a reporter gene detected by a light signal such as a fluorescence signal or a phosphorescence signal can correlate to the expression level of the reporter gene. This can be observed in a state in which a cell is maintained, and a cell used for evaluation can be easily selected, while the cell is alive. The reporter gene can be used in an experiment in which a test substance is continuously administered, and a change over time in the expression level of the reporter gene can be pursued in a real time.

In some embodiments, the first reporter gene and the second reporter gene are optically detectable. In some embodiments, the first reporter gene and the second reporter gene are optically distinguishable. In some embodiments, the first reporter gene and the second reporter gene are different reporter genes.

In some embodiments, the construct as disclosed herein comprises a polyadenylation signal (e.g. a poly-A tail) to effect proper polyadenylation of the gene transcript. Any poly-A sequence can be employed such as human or bovine growth hormone and SV40 polyadenylation signals and LTR polyadenylation signals. In an embodiment, the construct as disclosed herein comprises the polyadenylation signal from bovine growth hormone.

In certain embodiments, the construct as disclosed herein comprises an internal ribosome entry sites (IRES) element to create multigene, or polycistronic messages. IRES elements are able to bypass the ribosome-scanning model of 5′ methylated cap-dependent translation and begin translation at internal sites. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message. In an embodiment, the construct as disclosed herein comprises an IRES element from encephalomarcarditis virus.

The SARS2 replicon construct as described herein addresses the need for anti-SARS2 drug discovery and research on SARS2 and other viruses.

In some embodiments, the replicons described herein can be used for drug discovery. In other embodiments, the replicons described herein can be used as a surrogate platform for live SARS2 virus infection to understand the basics of SARS2 virology. Because of non-infectious nature of the replicon, it would be of great value to scientists who do not have access to the BSL3 facility that is required for working with SARS2.

In another aspect, this disclosure provides a method for screening a test compound for anti-SARS2 activity, wherein the method comprises: a) incubating the test compound with a cell line comprising the construct as described herein; and b) assaying for the presence of expression of the first reporter gene or expression of the second reporter gene, wherein a decreased expression of the first reporter gene or a decreased expression of the second reporter gene compared to a control indicates that the test compound comprises anti-SARS2 activity.

In order to determine the effect of a test compound on the SARS2 replicon construct, it will be necessary to transfer the SARS2 replicon construct as disclosed herein into a cell. Such transfer may employ viral or non-viral methods of gene transfer. A transformed cell comprising the SARS2 replicon construct is generated by introducing the SARS2 replicon construct into the cell. Suitable methods for polynucleotide delivery for transformation of an organelle, a cell, a tissue or an organism for use with the current methods include virtually any method by which a polynucleotide (e.g., DNA) can be introduced into an organelle, a cell, a tissue or an organism. A eukaryotic cell can be used as a recipient for the SARS2 replicon construct. Numerous cell lines and cultures are available for use as a host cell, and they can be obtained through the American Type Culture Collection (ATCC).

Any appropriate method may be used to transfect or transform the cells, or to administer the SARS2 replicon construct as disclosed herein. In some embodiments, examples of construct delivery into a cell can include methods such as delivery using cationic polymers and lipid like molecules.

The method for screening for a test compound for anti-SARS2 activity can comprise screening a test compound for its ability to inhibit or block the expression or function of the SARS2 replicon as disclosed herein. In the screening method as disclosed herein, a test compound having an ability to inhibit or block the expression or function of one or more SARS2 genes of the construct may be screened. When a substance having an effect of inhibiting the expression of one or more SARS2 genes of the construct, the test compound is capable of reducing the expression level of the one or more SARS2 genes of the construct by about 20%, about 25%, about 30%, about 35%, about 40%, about 45%, about 50%, about 55%, about 60%, about 70%, about 75%, about 80%, about 85%, about 90%, about 95% or more.

The cells to be used for screening are preferably cells of a biological species that is a host for SARS2 virus. In some embodiments, the cells are cultured cell lines of mammals and birds. In some embodiments, the cells are human cells. In some embodiments, the cells are avian cells.

The change in the expression level of the SARS2 genes of the construct can be evaluated using the reporter genes of the construct, or at the mRNA level or at the protein level. For example, the expression level of the SARS2 genes of the construct can be quantitatively compared by a nucleic acid amplification reaction such as RT-PCR method, immunohistochemistry method, or western blot (Western blot) method.

In another aspect, the disclosure also provides for a kit for use in screening a test compound for anti-SARS2 activity. In some embodiments, the kit comprises the replicon construct as disclosed herein. In certain embodiments, the kit comprises a replicon construct having the sequence of SEQ ID NO:14. In some embodiments, the kit can further comprise one or more primer/probe sets that provides a detectable signal on the occurrence on the transcription of one or more of the genes of the SARS2 replicon construct provided herein. In some embodiments, the kit can further comprise one or more cell lines to be transfected with the construct. In some embodiments, the kit further comprises one or more reagents for detection and quantitation of the two or more reporter genes of the replicon construct disclosed herein.

The design and organization of the replicon of the present disclosure can be used as a replicon for other viruses besides SARS2.

### EXAMPLES

Materials and Methods

Cells, transfection, and Remdesivir treatment. 293T and Vero were purchased from American Tissue Culture Collection (Manassas, VA) and were maintained in DMEM (Sigma-Aldrich, Burlington, MA) supplied with 10% FBS (Atlanta Biologicals, Flowery, GA) and penicillion/streptomycin in a 37° C., 5% CO₂ incubator. Cells were transfected with Lipofectamin 3000 (ThermoFisher Scientific, Waltham, MA) according to the manufacturer's protocol. For experiments involving Remdesivir treatment, the cells were treated with Remdesivir for 1 hour before transfection, continued with Remdesivir treatment for 24 hours following cell transfection with DNA or RNA, and harvested for the luciferase reporter gene assay, Western blotting, and RT-PCR analysis. Remdesivir was purchased from Cayman Chemical Company (Ann Arbor, MI) and dissolved in DMSO.

Synthesis of replicon fragments and construction of recombinant non-infectious SARS2 replicon DNA. The full-length SARS2 DNA replicon is 27,952 nucleotides (nt). It was synthesized in five fragments onto the pMX vector (for F2-5) or pMK vector (for F1/6) and sequenced by ThermoFisher Scientific. The sizes of the fragments were as follows: F2—nt 3583-8945; F3—nt 8942-15011; F4—nt 15007-21092; F5—nt 21078-24749; F1/6—nt 1-1642/nt 24689-27952. The fragments were designed with either BsaI or SalI restriction sites for subsequent cloning. Fragment F2-5 on pMX were ligated together at the unique BsaI sites using a Golden Gate Assembly kit (New England Biolabs, Ipswich, MA). Specifically, a ligation reaction (20 μl) containing 200 ng each of the four constructs (pMX.F2, 3, 4, or 5), T4 DNA ligase buffer and Golden Gate Enzyme Mix was set up and incubated at 37° C. for 1 hour to obtain the ligated product containing fragment 2-5 of the replicon on the pMX vector (pMX.F2-5). The ligated product was gel purified, digested with SalI, and gel purified to obtain fragment F2-5. pMK.F1/6 was digested with SalI and annealed with SalI-digested F2-5 through a pre-designed 50-nucleotide homolog overhang using a Gibson Assembly kit (New England Biolabs). Specifically, a reaction (20 μl) containing fragment F2-5, SalI-digested pMK.F1/6, and Gibson Assembly Master Mix was set up and incubated at 50° C. for 1 hour. An aliquot of the reaction was transformed into B10 competent E. coli (New England Biolabs). The final replicon DNA construct pMK.F1-6 was verified by PCR with 5 pairs of primers spanning each of the five adjacent junctions: 5′-aag atc gcc gtg taa gaa ttc cg-3 (nt 3315-3337; SEQ ID NO:01) and 5′-tgc ccg cgg tta tca tcg tgt t-3′ (nt 3919-3898; SEQ ID NO:02) for F1/F2; 5′-tgc ata gac ggt gct tta ctt ac-3′ (nt 8915-8937; SEQ ID NO:03) and 5′-ggt aca aga tca att ggt tgc tc-3′ (nt 9123-9101; SEQ ID NO:04) for F2/F3; 5′-ggt ggc aaa cct tgt atc aaa g-3′ (nt 14918-14939; SEQ ID NO:05) and 5′-gag gct ata gct tgt aag gtt gc-3′ (nt 15213-15191; SEQ ID NO:06) for F3/F4; 5′-cag ggc tca gaa tat gac tat g-3′ (nt 20956-20977; SEQ ID NO:07) and 5′-gtg tag gtg cct gtg tag gat-3′ (nt 21223-21206; SEQ ID NO:08) for F4/F5; 5′-ctt tgg ggt act gct gtt atg t-3′ (nt 24533-24554; SEQ ID NO:09) and 5′-cat ctc ctt cac ctt cac cag a-3′ (nt 24793-24772; SEQ ID NO:10) for F5/F6.

Purification of the SARS2 replicon plasmid DNA pMK.F1-6 and in vitro RNA transcription. A single colony was selected from the B10-transformed E. coli plate above, inoculated in 2 ml Amp+LB medium, and cultured at room temperature overnight at a shaker speed of 30 rpm. The 2 ml culture was transferred to 100 ml Amp+LB medium and continued to culture for 48 hours. The culture was pelleted and suspended 2 ml of 50 mM glucose, 50 mM Tris, pH 8.0, 10 mM EDTA, pH 8.0, and 50 μg/ml DNase-free RNase A, added 4 ml of 0.1 M NaOH and 1% SDS, incubated at room temperature for 3 minutes, added 3 ml of 1.5 M potassium acetate, pH 5.5, incubated at room temperature for 10 minutes. Mixing throughout the process had to be extremely gentle to avoid shearing of the large size plasmid DNA. Then, the mixture was spun at 3000 g for 10 minutes, the clear supernatant was recovered, added 2 volumes of isopropanol, incubated at −20° C. for 10 minutes, and spun at 10000 g for 10 minutes. The DNA pellet was rinsed with 75% ethanol, suspend in TE buffer containing 50 μg/ml DNase-free RNase A, incubated at room temperature for 30 minutes, and extracted with phenol/chloroform/isoamyl for 2-3 minutes. RNase A treatment and phenol/chloroform/isoamyl extraction were repeated two more times. The aqueous phase was added 2 volumes of isopropanol and spun at 10000 g for 10 minutes. The DNA pellet was rinsed with 75% ethanol, dried, and suspended in TE as the replicon plasmid pMK.F1-6 DNA. Throughout the process, all mixing steps had to be extremely gentle to avoid shearing of the plasmid DNA. For in vitro transcription, replicon RNA was synthesized using 10 μg replicon DNA/100 μl reaction and a T7 RoboMAX Express large-scale RNA synthesis kit (Promega, Madison, WI) in which Ribo m7G Cap analog (Promega) was included. The reaction was performed at 25° C. for 24 hours, treated with RQ1 RNase-Free DNase (Promega) at 37° C. 15 minutes, and extracted with phenol:chloroform:isoamyl once and chloroform:isoamyl once. The aqueous phase was added 2 volumes of isopropanol and spun at 10000 g for 10 minutes. The RNA pellet was rinsed with 75% ethanol, dried, and suspended in TE as replicon RNA.

Luciferase Reporter Gene Assay. 293T (1.5×10⁵ cells/well) and Vero (1.5×10⁵ cells/well) were plated in a 24-well plate, transfected with a total of 0.4 μg DNA or 0.3 μg RNA, cultured for 6-72 hours, harvested, and washed with PBS. The cells were lyzed and assayed for the luciferase activity using the Firefly Luciferase Assay system (Promega) according to the manufacturer's instructions and an Opticomp Luminometer (MGM Instruments, Hamden, CT).

Western blotting. 293T (4×10⁶ cells) were plated in a 10 cm plate, transfected with a total of 10 μg DNA or 7.5 μg RNA, cultured for 4-72 hours, harvested, and washed with PBS. The cells were lyzed in RIPA buffer (50 mM Tris-HCl, pH 7.4, 150 mM NaCl, 1% Triton X-100, 1% sodium deoxycholate, 0.1% SDS, 2 mm PMSF, and 1× protease inhibitor mixture (Roche, Indianapolis, IN) and incubated on ice for 20 minutes. The whole cell lysates (40 μg) were run 10% SDS-PAGE gel, blotted for direct detection of the GFP signal at 488 nm, or blotted against SARS2 nucleocapsid antibody (1:500; BEI Resources, Manassas, VA), followed by ECL visualization (ThermoFisher Scientific). Blots were stripped for re-probing against an anti-β-actin antibody (Sigma-Aldrich).

RT-PCR determination of (+) and (−) strand SARS2 replicon genomic RNA (gRNA) or N subgenomic RNA (sgRNA). 293T (6.5×10⁵ cells/well) were plated in a 6-well plate, transfected with a total of 1.5 μg DNA or 1.2 μg RNA, cultured for 24 hours, harvested, and washed with PBS. Total RNA was isolated from cells using a Trizol Reagents kit (ThermoFisher) according to the manufacturer's instructions, treated with RQ1 RNase-free DNase in 1×RQ1 DNase reaction buffer (Promega) at 37° C. 10 minutes, and extracted with an equal volume of acidic phenol (ThermoFisher Scientific). The aqueous phase was added 2 volumes of isopropanol and spun at 10000 g for 10 minutes. The RNA pellet was rinsed with 75% ethanol and suspended in RNase-free water (ThermoFisher Scientific). Primer TRS-L5′ was used to reverse transcribe (−) strand RNA to cDNA, while primer N3′ was used to reverse transcribe (+) strand RNA to cDNA. Primer pair N5′/N3′ was used to PCR amplify the full-length N gene (1260 bp) from both (+) and (−) strand gRNA and sgRNA-derived cDNA. Primer pair TRS-L5′/N3′ was used to PCR amplify both (+) and (−) strand sgRNA-derived cDNA. The sequences of the primers were as follows: TRS-L5′: 5′-atc tct tgt aga tct gtt ctc taa acg aac aaa cta aa-3′ (nt 845-874; SEQ ID NO:11); N-5′: 5′-atg tct gat aat gga ccc ca-3′ (nt 28274-29291; SEQ ID NO:12); N-3′: 5-tta ggc ctg agt tga gtc ag-3′ (nt 295534-29512; SEQ ID NO:13).

### Example 1: Design and Construction of the LTR/T7 Dual Promoter-Driven and GFP/fLuc Dual Reporter-Expressing SARS2 Replicon

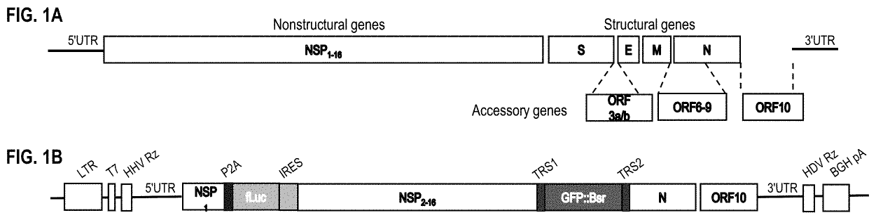

Using as the reference the Wuhan-Hu-1 isolate SARS2 genome (GenBank: NC_045512.2, FIG. 1A), the two-in-one SARS replicon was engineered to have both HIV LTR promoter and T7 promoter at the 5′ end (FIG. 1B). When co-expressed with HIV Tat protein in cells, the DNA replicon would ensure transcription of the unusually large full-length viral RNA replicon through the LTR promoter [44-47]. Alternatively, the DNA replicon could be used as the template to synthesize the RNA replicon using a T7 DNA-dependent RNA polymerase-based in vitro transcription kit, which often gave rise to a limited amount of the full-length RNA of this large size. A few other features have been incorporated into the replicon (Table 1).

TABLE 1
Replicon Features

| Feature | Abbreviation | Location | Size (bp) | Function |
|---|---|---|---|---|
| HIV-1 LTR promoter | LTR | 1-713 | 713 | To facilitate expression of long |
| | | | | RNA transcript when |
| | | | | transfected with HIV Tat |
| T7 promoter | T7 | 722-740 | 19 | To synthesize viral RNA by in |
| | | | | vitro transcription |
| Hammerhead virus | HHV Rz | 740-799 | 59 | To produce the native 5′ end of |
| ribozyme | | | | SARS2 viral RNA genome |
| SARS2 5′ | 5′UTR | 800-1064 | 265 | To maintain the regulatory |
| untranslated region | | | | element for SARS2 replication |
| SARS2 nonstructural | NSP1 | 1065-1604 | 549 | To encode NSP1 |
| protein 1 | | | | |
| Porcine teschovirus-1 | P2A | 1611-1671 | 66 | To cleave NSP1-fLuc fusion |
| self-cleaving peptide | | | | protein to ensure proper fLuc |
| 2A | | | | expression |
| Firefly luciferase | fLuc | 1677-3329 | 1653 | To monitor translation from and |
| | | | | replication of SARS2 RNA |
| Encephalomarcarditis | IRES | 3330-3910 | 581 | To facilitate translation of the |
| virus internal | | | | long transcript of SARS2 NSP2- |
| ribozyme entry site | | | | 16 |
| SARS2 nonstructural | NSP2-16 | 3917-24666 | 20750 | To encode nonstructural SARS |
| protein 2-16 | | | | proteins NSP2-16 for replication |
| Transcriptional | TRS1 | 24675-24681 | 7 | To maintain the authentic |
| regulatory sequence | | | | regulatory element for GFP:Bsr |
| 1 | | | | expression |
| Green fluorescent | GFP:Bsr | 24682-25998 | 1317 | To select stable SARS2 |
| protein-blastidin | | | | replicon and monitor SARS2 |
| fusion protein | | | | replicon replication |
| Transcriptional | TRS2 | 25999-26102 | 14 | To maintain the authentic |
| regulatory sequence | | | | regulatory element for SARS2 |
| 2 | | | | nucleocapsid expression |
| SARS2 nucleocapsid | N | 26013-27272 | 1260 | To encode SARS2 |
| protein | | | | nucleocapsid and monitor |
| | | | | SARS2 replicon replication |
| SARS2 ORF10-3′ | ORF10- | 27373-27642 | 370 | To maintain the regulatory |
| untranslated region | 3′UTR | | | element integrity for SARS2 |
| | | | | replication |
| Hepatitis delta virus | HDV Rz | 27643-27721 | 79 | To produce the native 3′ end of |
| ribozyme | | | | SARS2 viral RNA genome |
| Bovine growth | BGH pA | 27728-27952 | 225 | To stabilize the RNA transcript |
| hormone | | | | |
| polyadenylation | | | | |
| signal | | | | |

TABLE 2
Replicon sequence
Nucleic acid sequence of replicon (SEQ ID NO: 14)

ACCTGGAAAAACATGGAGCAATCACAAGTAGCAATACAGCAGCTACCAATGCTGCTTGTGCCTGGCTAGAAGCACA
AGAGGAGGAGGAGGTGGGTTTTCCAGTCACACCTCAGGTACCTTTAAGACCAATGACTTACAAGGCAGCTGTAGAT
CTTAGCCACTTTTTAAAAGAAAAGGGGGGACTGGAAGGGCTAATTCACTCCCAACGAAGACAAGATATCCTTGATC
TGTGGATCTACCACACACAAGGCTACTTCCCTGATTGGCAGAACTACACACCAGGGCCAGGGGTCAGATATCCACT
GACCTTTGGATGGTGCTACAAGCTAGTACCAGTTGAGCCAGATAAGGTAGAAGAGGCCAATAAAGGAGAGAACACC
AGCTTGTTACACCCTGTGAGCCTGCATGGAATGGATGACCCTGAGAGAGAAGTGTTAGAGTGGAGGTTTGACAGCC
GCCTAGCATTTCATCACGTGGCCCGAGAGCTGCATCCGGAGTACTTCAAGAACTGCTGACATCGAGCTTGCTACAA
GGGACTTTCCGCTGGGGACTTTCCAGGGAGGCGTGGCCTGGGCGGGACTGGGGAGTGGCGAGCCCTCAGATGCTGC
ATATAAGCAGCTGCTTTTTGCCTGTACTGGGTCTCTCTGGTTAGACCAGATTTGAGCCTGGGAGCTCTCTGGCTAA
CTAGGGAACCCACTGCTTAAGCCTCAATAGGCCGGCCTAATACGACTCACTATAGGGGGGAGAGGCCCGAAGGCCC
TGATGAGTCCGTGAGGACGAAACGGTACCCGGTACCGTCATTAAAGGTTTATACCTTCCCAGGTAACAAACCAACC
AACTTTCGATCTCTTGTAGATCTGTTCTCTAAACGAACTTTAAAATCTGTGTGGCTGTCACTCGGCTGCATGCTTA
GTGCACTCACGCAGTATAATTAATAACTAATTACTGTCGTTGACAGGACACGAGTAACTCGTCTATCTTCTGCAGG
CTGCTTACGGTTTCGTCCGTGTTGCAGCCGATCATCAGCACATCTAGGTTTCGTCCGGGTGTGACCGAAAGGTAAG
ATGGAGAGCCTTGTCCCTGGTTTCAACGAGAAAACACACGTCCAACTCAGTTTGCCTGTTTTACAGGTTCGCGACG
TGCTCGTACGTGGCTTTGGAGACTCCGTGGAGGAGGTCTTATCAGAGGCACGTCAACATCTTAAAGATGGCACTTG
TGGCTTAGTAGAAGTTGAAAAAGGCGTTTTGCCTCAACTTGAACAGCCCTATGTGTTCATCAAACGTTCGGATGCT
CGAACTGCACCTCATGGTCATGTTATGGTTGAGCTGGTAGCAGAACTCGAAGGCATTCAGTACGGTCGTAGTGGTG
AGACACTTGGTGTCCTTGTCCCTCATGTGGGCGAAATACCAGTGGCTTACCGCAAGGTTCTTCTTCGTAAGAACGG
TAATAAAGGAGCTGGTGGCCATAGTTACGGCGCCGATCTAAAGTCATTTGACTTAGGCGACGAGCTTGGCACTGAT
CCTTATGAAGATTTTCAAGAAAACTGGAACACTAAACATAGCAGTGGTGTTACCCGTGAACTCATGCGTGAGCTTA
ACGGAGGGCCGCGGGGAAGCGGAGCTACTAACTTCAGCCTGCTGAAGCAGGCTGGAGACGTGGAGGAGAACCCTGG
ACCTATGGAAGATGCCAAAAACATTAAGAAGGGCCCAGCGCCATTCTACCCACTCGAAGACGGGACCGCCGGCGAG
CAGCTGCACAAAGCCATGAAGCGCTACGCCCTGGTGCCCGGCACCATCGCCTTTACCGACGCACATATCGAGGTGG
ACATTACCTACGCCGAGTACTTCGAGATGAGCGTTCGGCTGGCAGAAGCTATGAAGCGCTATGGGCTGAATACAAA
CCATCGGATCGTGGTGTGCAGCGAGAATAGCTTGCAGTTCTTCATGCCCGTGTTGGGTGCCCTGTTCATCGGTGTG
GCTGTGGCCCCAGCTAACGACATCTACAACGAGCGCGAGCTGCTGAACAGCATGGGCATCAGCCAGCCCACCGTCG
TATTCGTGAGCAAGAAAGGGCTGCAAAAGATCCTCAACGTGCAAAAGAAGCTACCGATCATACAAAAGATCATCAT
CATGGATAGCAAGACCGACTACCAGGGCTTCCAAAGCATGTACACCTTCGTGACTTCCCATTTGCCACCCGGCTTC
AACGAGTACGACTTCGTGCCCGAGAGCTTCGACCGGGACAAAACCATCGCCCTGATCATGAACAGTAGTGGCAGTA
CCGGATTGCCCAAGGGCGTAGCCCTACCGCACCGCACCGCTTGTGTCCGATTCAGTCATGCCCGCGACCCCATCTT
CGGCAACCAGATCATCCCCGACACCGCTATCCTCAGCGTGGTGCCATTTCACCACGGCTTCGGCATGTTCACCACG
CTGGGCTACTTGATCTGCGGCTTTCGGGTCGTGCTCATGTACCGCTTCGAGGAGGAGCTATTCTTGCGCAGCTTGC
AAGACTATAAGATTCAATCTGCCCTGCTGGTGCCCACACTATTTAGCTTCTTCGCTAAGAGCACTCTCATCGACAA
GTACGACCTAAGCAACTTGCACGAGATCGCCAGCGGCGGGGCGCCGCTCAGCAAGGAGGTAGGTGAGGCCGTGGCC
AAACGCTTCCACCTACCAGGCATCCGCCAGGGCTACGGCCTGACAGAAACAACCAGCGCCATTCTGATCACCCCCG
AAGGGGACGACAAGCCTGGCGCAGTAGGCAAGGTGGTGCCCTTCTTCGAGGCTAAGGTGGTGGACTTGGACACCGG
TAAGACACTGGGTGTGAACCAGCGCGGCGAGCTGTGCGTCCGTGGCCCCATGATCATGAGCGGCTACGTTAACAAC
CCCGAGGCTACAAACGCTCTCATCGACAAGGACGGCTGGCTGCACAGCGGCGACATCGCCTACTGGGACGAGGACG
AGCACTTCTTCATCGTGGACCGGCTGAAGAGCCTGATCAAATACAAGGGCTACCAGGTAGCCCCAGCCGAACTGGA
GAGCATCCTGCTGCAACACCCCAACATCTTCGACGCCGGGGTCGCCGGCCTGCCCGACGACGATGCCGGCGAGCTG
CCCGCCGCAGTCGTCGTGCTGGAACACGGTAAAACCATGACCGAGAAGGAGATCGTGGACTATGTGGCCAGCCAGG
TTACAACCGCCAAGAAGCTGCGCGGTGGTGTTGTGTTCGTGGACGAGGTGCCTAAAGGACTGACCGGCAAGTTGGA
CGCCCGCAAGATCCGCGAGATTCTCATTAAGGCCAAGAAGGGCGGCAAGATCGCCGTGTAAGAATTCCGCCCCTCT
CCCTCCCCCCCCCCTAACGTTACTGGCCGAAGCCGCTTGGAATAAGGCCGGTGTGCGTTTGTCTATATGTTATTTT
CCACCATATTGCCGTCTTTTGGCAATGTGAGGGCCCGGAAACCTGGCCCTGTCTTCTTGACGAGCATTCCTAGGGG
TCTTTCCCCTCTCGCCAAAGGAATGCAAGGTCTGTTGAATGTCGTGAAGGAAGCAGTTCCTCTGGAAGCTTCTTGA
AGACAAACAACGTCTGTAGCGACCCTTTGCAGGCAGCGGAACCCCCCACCTGGCGACAGGTGCCTCTGCGGCCAAA
AGCCACGTGTATAAGATACACCTGCAAAGGCGGCACAACCCCAGTGCCACGTTGTGAGTTGGATAGTTGTGGAAAG
AGTCAAATGGCTCTCCTCAAGCGTATTCAACAAGGGGCTGAAGGATGCCCAGAAGGTACCCCATTGTATGGGATCT
GATCTGGGGCCTCGGTGCACATGCTTTACATGTGTTTAGTCGAGGTTAAAAAACGTCTAGGCCCCCCGAACCACGG
GGACGTGGTTTTCCTTTGAAAAACACGATGATAACCGCGGGCATACACTCGCTATGTCGATAACAACTTCTGTGGC
CCTGATGGCTACCCTCTTGAGTGCATTAAAGACCTTCTAGCACGTGCTGGTAAAGCTTCATGCACTTTGTCCGAAC
AACTGGACTTTATTGACACTAAGAGGGGTGTATACTGCTGCCGTGAACATGAGCATGAAATTGCTTGGTACACGGA
ACGTTCTGAAAAGAGCTATGAATTGCAGACACCTTTTGAAATTAAATTGGCAAAGAAATTTGACACCTTCAATGGG
GAATGTCCAAATTTTGTATTTCCCTTAAATTCCATAATCAAGACTATTCAACCAAGGGTTGAAAAGAAAAAGCTTG
ATGGCTTTATGGGTAGAATTCGATCTGTCTATCCAGTTGCGTCACCAAATGAATGCAACCAAATGTGCCTTTCAAC
TCTCATGAAGTGTGATCATTGTGGTGAAACTTCATGGCAGACGGGCGATTTTGTTAAAGCCACTTGCGAATTTTGT
GGCACTGAGAATTTGACTAAAGAAGGTGCCACTACTTGTGGTTACTTACCCCAAAATGCTGTTGTTAAAATTTATT
GTCCAGCATGTCACAATTCAGAAGTAGGACCTGAGCATAGTCTTGCCGAATACCATAATGAATCTGGCTTGAAAAC
CATTCTTCGTAAGGGTGGTCGCACTATTGCCTTTGGAGGCTGTGTGTTCTCTTATGTTGGTTGCCATAACAAGTGT
GCCTATTGGGTTCCACGTGCTAGCGCTAACATAGGTTGTAACCATACAGGTGTTGTTGGAGAAGGTTCCGAAGGTC
TTAATGACAACCTTCTTGAAATACTCCAAAAAGAGAAAGTCAACATCAATATTGTTGGTGACTTTAAACTTAATGA
AGAGATCGCCATTATTTTGGCATCTTTTTCTGCTTCCACAAGTGCTTTTGTGGAAACTGTGAAAGGTTTGGATTAT
AAAGCATTCAAACAAATTGTTGAATCCTGTGGTAATTTTAAAGTTACAAAAGGAAAAGCTAAAAAAGGTGCCTGGA
ATATTGGTGAACAGAAATCAATACTGAGTCCTCTTTATGCATTTGCATCAGAGGCTGCTCGTGTTGTACGATCAAT
TTTCTCCCGCACTCTTGAAACTGCTCAAAATTCTGTGCGTGTTTTACAGAAGGCCGCTATAACAATACTAGATGGA
ATTTCACAGTATTCACTGAGACTCATTGATGCTATGATGTTCACATCTGATTTGGCTACTAACAATCTAGTTGTAA
TGGCCTACATTACAGGTGGTGTTGTTCAGTTGACTTCGCAGTGGCTAACTAACATCTTTGGCACTGTTTATGAAAA
ACTCAAACCCGTCCTTGATTGGCTTGAAGAGAAGTTTAAGGAAGGTGTAGAGTTTCTTAGAGACGGTTGGGAAATT
GTTAAATTTATCTCAACCTGTGCTTGTGAAATTGTCGGTGGACAAATTGTCACCTGTGCAAAGGAAATTAAGGAGA
GTGTTCAGACATTCTTTAAGCTTGTAAATAAATTTTTGGCTTTGTGTGCTGACTCTATCATTATTGGTGGAGCTAA
ACTTAAAGCCTTGAATTTAGGTGAAACATTTGTCACGCACTCAAAGGGATTGTACAGAAAGTGTGTTAAATCCAGA
GAAGAAACTGGCCTACTCATGCCTCTAAAAGCCCCAAAAGAAATTATCTTCTTAGAGGGAGAAACACTTCCCACAG
AAGTGTTAACAGAGGAAGTTGTCTTGAAAACTGGTGATTTACAACCATTAGAACAACCTACTAGTGAAGCTGTTGA
AGCTCCATTGGTTGGTACACCAGTTTGTATTAACGGGCTTATGTTGCTCGAAATCAAAGACACAGAAAAGTACTGT
GCCCTTGCACCTAATATGATGGTAACAAACAATACCTTCACACTCAAAGGCGGTGCACCAACAAAGGTTACTTTTG
GTGATGACACTGTGATAGAAGTGCAAGGTTACAAGAGTGTGAATATCACTTTTGAACTTGATGAAAGGATTGATAA
AGTACTTAATGAGAAGTGCTCTGCCTATACAGTTGAACTCGGTACAGAAGTAAATGAGTTCGCCTGTGTTGTGGCA
GATGCTGTCATAAAAACTTTGCAACCAGTATCTGAATTACTTACACCACTGGGCATTGATTTAGATGAGTGGAGTA
TGGCTACATACTACTTATTTGATGAGTCTGGTGAGTTTAAATTGGCTTCACATATGTATTGTTCTTTCTACCCTCC
AGATGAGGATGAAGAAGAAGGTGATTGTGAAGAAGAAGAGTTTGAGCCATCAACTCAATATGAGTATGGTACTGAA
GATGATTACCAAGGTAAACCTTTGGAATTTGGTGCCACTTCTGCTGCTCTTCAACCTGAAGAAGAGCAAGAAGAAG
ATTGGTTAGATGATGATAGTCAACAAACTGTTGGTCAACAAGACGGCAGTGAGGACAATCAGACAACTACTATTCA
AACAATTGTTGAGGTTCAACCTCAATTAGAGATGGAACTTACACCAGTTGTTCAGACTATTGAAGTGAATAGTTTT
AGTGGTTATTTAAAACTTACTGACAATGTATACATTAAAAATGCAGACATTGTGGAAGAAGCTAAAAAGGTAAAAC
CAACAGTGGTTGTTAATGCAGCCAATGTTTACCTTAAACATGGAGGAGGTGTTGCAGGAGCCTTAAATAAGGCTAC
TAACAATGCCATGCAAGTTGAATCTGATGATTACATAGCTACTAATGGACCACTTAAAGTGGGTGGTAGTTGTGTT
TTAAGCGGACACAATCTTGCTAAACACTGTCTTCATGTTGTCGGCCCAAATGTTAACAAAGGTGAAGACATTCAAC
TTCTTAAGAGTGCTTATGAAAATTTTAATCAGCACGAAGTTCTACTTGCACCATTATTATCAGCTGGTATTTTTGG
TGCTGACCCTATACATTCTTTAAGAGTTTGTGTAGATACTGTTCGCACAAATGTCTACTTAGCTGTCTTTGATAAA
AATCTCTATGACAAACTTGTTTCAAGCTTTTTGGAAATGAAGAGTGAAAAGCAAGTTGAACAAAAGATCGCTGAGA
TTCCTAAAGAGGAAGTTAAGCCATTTATAACTGAAAGTAAACCTTCAGTTGAACAGAGAAAACAAGATGATAAGAA
AATCAAAGCTTGTGTTGAAGAAGTTACAACAACTCTGGAAGAAACTAAGTTCCTCACAGAAAACTTGTTACTTTAT
ATTGACATTAATGGCAATCTTCATCCAGATTCTGCCACTCTTGTTAGTGACATTGACATCACTTTCTTAAAGAAAG
ATGCTCCATATATAGTGGGTGATGTTGTTCAAGAGGGTGTTTTAACTGCTGTGGTTATACCTACTAAAAAGGCTGG
TGGCACTACTGAAATGCTAGCGAAAGCTTTGAGAAAAGTGCCAACAGACAATTATATAACCACTTACCCGGGTCAG
GGTTTAAATGGTTACACTGTAGAGGAGGCAAAGACAGTGCTTAAAAAGTGTAAAAGTGCCTTTTACATTCTACCAT
CTATTATCTCTAATGAGAAGCAAGAAATTCTTGGAACTGTTTCTTGGAATTTGCGAGAAATGCTTGCACATGCAGA
AGAAACACGCAAATTAATGCCTGTCTGTGTGGAAACTAAAGCCATAGTTTCAACTATACAGCGTAAATATAAGGGT
ATTAAAATACAAGAGGGTGTGGTTGATTATGGTGCTAGATTTTACTTTTACACCAGTAAAACAACTGTAGCGTCAC
TTATCAACACACTTAACGATCTAAATGAAACTCTTGTTACAATGCCACTTGGCTATGTAACACATGGCTTAAATTT
GGAAGAAGCTGCTCGGTATATGAGATCTCTCAAAGTGCCAGCTACAGTTTCTGTTTCTTCACCTGATGCTGTTACA
GCGTATAATGGTTATCTTACTTCTTCTTCTAAAACACCTGAAGAACATTTTATTGAAACCATCTCACTTGCTGGTT
CCTATAAAGATTGGTCCTATTCTGGACAATCTACACAACTAGGTATAGAATTTCTTAAGAGAGGTGATAAAAGTGT
ATATTACACTAGTAATCCTACCACATTCCACCTAGATGGTGAAGTTATCACCTTTGACAATCTTAAGACACTTCTT
TCTTTGAGAGAAGTGAGGACTATTAAGGTGTTTACAACAGTAGACAACATTAACCTCCACACGCAAGTTGTGGACA
TGTCAATGACATATGGACAACAGTTTGGTCCAACTTATTTGGATGGAGCTGATGTTACTAAAATAAAACCTCATAA
TTCACATGAAGGTAAAACATTTTATGTTTTACCTAATGATGACACTCTACGTGTTGAGGCTTTTGAGTACTACCAC
ACAACTGATCCTAGTTTTCTGGGTAGGTACATGTCAGCATTAAATCACACTAAAAAGTGGAAATACCCACAAGTTA
ATGGTTTAACTTCTATTAAATGGGCAGATAACAACTGTTATCTTGCCACTGCATTGTTAACACTCCAACAAATAGA
GTTGAAGTTTAATCCACCTGCTCTACAAGATGCTTATTACAGAGCAAGGGCTGGTGAAGCTGCTAACTTTTGTGCA
CTTATCTTAGCCTACTGTAATAAGACAGTAGGTGAGTTAGGTGATGTTAGAGAAACAATGAGTTACTTGTTTCAAC
ATGCCAATTTAGATTCTTGCAAAAGAGTCTTGAACGTGGTGTGTAAAACTTGTGGACAACAGCAGACAACCCTTAA
GGGTGTAGAAGCTGTTATGTACATGGGCACACTTTCTTATGAACAATTTAAGAAAGGTGTTCAGATACCTTGTACG
TGTGGTAAACAAGCTACAAAATATCTAGTACAACAGGAGTCACCTTTTGTTATGATGTCAGCACCACCTGCTCAGT
ATGAACTTAAGCATGGTACATTTACTTGTGCTAGTGAGTACACTGGTAATTACCAGTGTGGTCACTATAAACATAT
AACTTCTAAAGAAACTTTGTATTGCATAGACGGTGCTTTACTTACAAAGTCCTCAGAATACAAAGGTCCTATTACG
GATGTTTTCTACAAAGAAAACAGTTACACAACAACCATAAAACCAGTTACTTATAAATTGGATGGTGTTGTTTGTA
CAGAAATTGACCCTAAGTTGGACAATTATTATAAGAAAGACAATTCTTATTTCACAGAGCAACCAATTGATCTTGT
ACCAAACCAACCATATCCAAACGCAAGCTTCGATAATTTTAAGTTTGTATGTGATAATATCAAATTTGCTGATGAT
TTAAACCAGTTAACTGGTTATAAGAAACCTGCTTCAAGAGAGCTTAAAGTTACATTTTTCCCTGACTTAAATGGTG
ATGTGGTGGCTATTGATTATAAACACTACACACCCTCTTTTAAGAAAGGAGCTAAATTGTTACATAAACCTATTGT
TTGGCATGTTAACAATGCAACTAATAAAGCCACGTATAAACCAAATACCTGGTGTATACGTTGTCTTTGGAGCACA
AAACCAGTTGAAACATCAAATTCGTTTGATGTACTGAAGTCAGAGGACGCGCAGGGAATGGATAATCTTGCCTGCG
AAGATCTAAAACCAGTCTCTGAAGAAGTAGTGGAAAATCCTACCATACAGAAAGACGTTCTTGAGTGTAATGTGAA
AACTACCGAAGTTGTAGGAGACATTATACTTAAACCAGCAAATAATAGTTTAAAAATTACAGAAGAGGTTGGCCAC
ACAGATCTAATGGCTGCTTATGTAGACAATTCTAGTCTTACTATTAAGAAACCTAATGAATTATCTAGAGTATTAG
GTTTGAAAACCCTTGCTACTCATGGTTTAGCTGCTGTTAATAGTGTCCCTTGGGATACTATAGCTAATTATGCTAA
GCCTTTTCTTAACAAAGTTGTTAGTACAACTACTAACATAGTTACACGGTGTTTAAACCGTGTTTGTACTAATTAT
ATGCCTTATTTCTTTACTTTATTGCTACAATTGTGTACTTTTACTAGAAGTACAAATTCTAGAATTAAAGCATCTA
TGCCGACTACTATAGCAAAGAATACTGTTAAGAGTGTCGGTAAATTTTGTCTAGAGGCTTCATTTAATTATTTGAA
GTCACCTAATTTTTCTAAACTGATAAATATTATAATTTGGTTTTTACTATTAAGTGTTTGCCTAGGTTCTTTAATC
TACTCAACCGCTGCTTTAGGTGTTTTAATGTCTAATTTAGGCATGCCTTCTTACTGTACTGGTTACAGAGAAGGCT
ATTTGAACTCTACTAATGTCACTATTGCAACCTACTGTACTGGTTCTATACCTTGTAGTGTTTGTCTTAGTGGTTT
AGATTCTTTAGACACCTATCCTTCTTTAGAAACTATACAAATTACCATTTCATCTTTTAAATGGGATTTAACTGCT
TTTGGCTTAGTTGCAGAGTGGTTTTTGGCATATATTCTTTTCACTAGGTTTTTCTATGTACTTGGATTGGCTGCAA
TCATGCAATTGTTTTTCAGCTATTTTGCAGTACATTTTATTAGTAATTCTTGGCTTATGTGGTTAATAATTAATCT
TGTACAAATGGCCCCGATTTCAGCTATGGTTAGAATGTACATCTTCTTTGCATCATTTTATTATGTATGGAAAAGT
TATGTGCATGTTGTAGACGGTTGTAATTCATCAACTTGTATGATGTGTTACAAACGTAATAGAGCAACAAGAGTCG
AATGTACAACTATTGTTAATGGTGTTAGAAGGTCCTTTTATGTCTATGCTAATGGAGGTAAAGGCTTTTGCAAACT
ACACAATTGGAATTGTGTTAATTGTGATACATTCTGTGCTGGTAGTACATTTATTAGTGATGAAGTTGCGAGAGAC
TTGTCACTACAGTTTAAAAGACCAATAAATCCTACTGACCAGTCTTCTTACATCGTTGATAGTGTTACAGTGAAGA
ATGGTTCCATCCATCTTTACTTTGATAAAGCTGGTCAAAAGACTTATGAAAGACATTCTCTCTCTCATTTTGTTAA
CTTAGACAACCTGAGAGCTAATAACACTAAAGGTTCATTGCCTATTAATGTTATAGTTTTTGATGGTAAATCAAAA
TGTGAAGAATCATCTGCAAAATCAGCGTCTGTTTACTACAGTCAGCTTATGTGTCAACCTATACTGTTACTAGATC
AGGCATTAGTGTCTGATGTTGGTGATAGTGCGGAAGTTGCAGTTAAAATGTTTGATGCTTACGTTAATACGTTTTC
ATCAACTTTTAACGTACCAATGGAAAAACTCAAAACACTAGTTGCAACTGCAGAAGCTGAACTTGCAAAGAATGTG
TCCTTAGACAATGTCTTATCTACTTTTATTTCAGCAGCTCGGCAAGGGTTTGTTGATTCAGATGTAGAAACTAAAG
ATGTTGTTGAATGTCTTAAATTGTCACATCAATCTGACATAGAAGTTACTGGCGATAGTTGTAATAACTATATGCT
CACCTATAACAAAGTTGAAAACATGACACCCCGTGACCTTGGTGCTTGTATTGACTGTAGTGCGCGTCATATTAAT
GCGCAGGTAGCAAAAAGTCACAACATTGCTTTGATATGGAACGTTAAAGATTTCATGTCATTGTCTGAACAACTAC
GAAAACAAATACGTAGTGCTGCTAAAAAGAATAACTTACCTTTTAAGTTGACATGTGCAACTACTAGACAAGTTGT
TAATGTTGTAACAACAAAGATAGCACTTAAGGGTGGTAAAATTGTTAATAATTGGTTGAAGCAGTTAATTAAAGTT
ACACTTGTGTTCCTTTTTGTTGCTGCTATTTTCTATTTAATAACACCTGTTCATGTCATGTCTAAACATACTGACT
TTTCAAGTGAAATCATAGGATACAAGGCTATTGATGGTGGTGTCACTCGTGACATAGCATCTACAGATACTTGTTT
TGCTAACAAACATGCTGATTTTGACACATGGTTTAGCCAGCGTGGTGGTAGTTATACTAATGACAAAGCTTGCCCA
TTGATTGCTGCAGTCATAACAAGAGAAGTGGGTTTTGTCGTGCCTGGTTTGCCTGGCACGATATTACGCACAACTA
ATGGTGACTTTTTGCATTTCTTACCTAGAGTTTTTAGTGCAGTTGGTAACATCTGTTACACACCATCAAAACTTAT
AGAGTACACTGACTTTGCAACATCAGCTTGTGTTTTGGCTGCTGAATGTACAATTTTTAAAGATGCTTCTGGTAAG
CCAGTACCATATTGTTATGATACCAATGTACTAGAAGGTTCTGTTGCTTATGAAAGTTTACGCCCTGACACACGTT
ATGTGCTCATGGATGGCTCTATTATTCAATTTCCTAACACCTACCTTGAAGGTTCTGTTAGAGTGGTAACAACTTT
TGATTCTGAGTACTGTAGGCACGGCACTTGTGAAAGATCAGAAGCTGGTGTTTGTGTATCTACTAGTGGTAGATGG
GTACTTAACAATGATTATTACAGATCTTTACCAGGAGTTTTCTGTGGTGTAGATGCTGTAAATTTACTTACTAATA
TGTTTACACCACTAATTCAACCTATTGGTGCTTTGGACATATCAGCATCTATAGTAGCTGGTGGTATTGTAGCTAT
CGTAGTAACATGCCTTGCCTACTATTTTATGAGGTTTAGAAGAGCTTTTGGTGAATACAGTCATGTAGTTGCCTTT
AATACTTTACTATTCCTTATGTCATTCACTGTACTCTGTTTAACACCAGTTTACTCATTCTTACCTGGTGTTTATT
CTGTTATTTACTTGTACTTGACATTTTATCTTACTAATGATGTTTCTTTTTTAGCACATATTCAGTGGATGGTTAT
GTTCACACCTTTAGTACCTTTCTGGATAACAATTGCTTATATCATTTGTATTTCCACAAAGCATTTCTATTGGTTC
TTTAGTAATTACCTAAAGAGACGTGTAGTCTTTAATGGTGTTTCCTTTAGTACTTTTGAAGAAGCTGCGCTGTGCA
CCTTTTTGTTAAATAAAGAAATGTATCTAAAGTTGCGTAGTGATGTGCTATTACCTCTTACGCAATATAATAGATA
CTTAGCTCTTTATAATAAGTACAAGTATTTTAGTGGAGCAATGGATACAACTAGCTACAGAGAAGCTGCTTGTTGT
CATCTCGCAAAGGCTCTCAATGACTTCAGTAACTCAGGTTCTGATGTTCTTTACCAACCACCACAAACCTCTATCA
CCTCAGCTGTTTTGCAGAGTGGTTTTAGAAAAATGGCATTCCCATCTGGTAAAGTTGAGGGTTGTATGGTACAAGT
AACTTGTGGTACAACTACACTTAACGGTCTTTGGCTTGATGACGTAGTTTACTGTCCAAGACATGTGATCTGCACC
TCTGAAGACATGCTTAACCCTAATTATGAAGATTTACTCATTCGTAAGTCTAATCATAATTTCTTGGTACAGGCTG
GTAATGTTCAACTCAGGGTTATTGGACATTCTATGCAAAATTGTGTACTTAAGCTTAAGGTTGATACAGCCAATCC
TAAGACACCTAAGTATAAGTTTGTTCGCATTCAACCAGGACAGACTTTTTCAGTGTTAGCTTGTTACAATGGTTCA
CCATCTGGTGTTTACCAATGTGCTATGAGGCCCAATTTCACTATTAAGGGTTCATTCCTTAATGGTTCATGTGGTA
GTGTTGGTTTTAACATAGATTATGACTGTGTCTCTTTTTGTTACATGCACCATATGGAATTACCAACTGGAGTTCA
TGCTGGCACAGACTTAGAAGGTAACTTTTATGGACCTTTTGTTGACAGGCAAACAGCACAAGCAGCTGGTACGGAC
ACAACTATTACAGTTAATGTTTTAGCTTGGTTGTACGCTGCTGTTATAAATGGAGACAGGTGGTTTCTCAATCGAT
TTACCACAACTCTTAATGACTTTAACCTTGTGGCTATGAAGTACAATTATGAACCTCTAACACAAGACCATGTTGA
CATACTAGGACCTCTTTCTGCTCAAACTGGAATTGCCGTTTTAGATATGTGTGCTTCATTAAAAGAATTACTGCAA
AATGGTATGAATGGACGTACCATATTGGGTAGTGCTTTATTAGAAGATGAATTTACACCTTTTGATGTTGTTAGAC
AATGCTCAGGTGTTACTTTCCAAAGTGCAGTGAAAAGAACAATCAAGGGTACACACCACTGGTTGTTACTCACAAT
TTTGACTTCACTTTTAGTTTTAGTCCAGAGTACTCAATGGTCTTTGTTCTTTTTTTTGTATGAAAATGCCTTTTTA
CCTTTTGCTATGGGTATTATTGCTATGTCTGCTTTTGCAATGATGTTTGTCAAACATAAGCATGCATTTCTCTGTT
TGTTTTTGTTACCTTCTCTTGCCACTGTAGCTTATTTTAATATGGTCTATATGCCTGCTAGTTGGGTGATGCGTAT
TATGACATGGTTGGATATGGTTGATACTAGTTTGTCTGGTTTTAAGCTAAAAGACTGTGTTATGTATGCATCAGCT
GTAGTGTTACTAATCCTTATGACAGCAAGAACTGTGTATGATGATGGTGCTAGGAGAGTGTGGACACTTATGAATG
TCTTGACACTCGTTTATAAAGTTTATTATGGTAATGCTTTAGATCAAGCCATTTCCATGTGGGCTCTTATAATCTC
TGTTACTTCTAACTACTCAGGTGTAGTTACAACTGTCATGTTTTTGGCCAGAGGTATTGTTTTTATGTGTGTTGAG
TATTGCCCTATTTTCTTCATAACTGGTAATACACTTCAGTGTATAATGCTAGTTTATTGTTTCTTAGGCTATTTTT
GTACTTGTTACTTTGGCCTCTTTTGTTTACTCAACCGCTACTTTAGACTGACTCTTGGTGTTTATGATTACTTAGT
TTCTACACAGGAGTTTAGATATATGAATTCACAGGGACTACTCCCACCCAAGAATAGCATAGATGCCTTCAAACTC
AACATTAAATTGTTGGGTGTTGGTGGCAAACCTTGTATCAAAGTAGCCACTGTACAGTCTAAAATGTCAGATGTAA
AGTGCACATCAGTAGTCTTACTCTCAGTTTTGCAACAACTCAGAGTAGAATCATCATCTAAATTGTGGGCTCAATG
TGTCCAGTTACACAATGACATTCTCTTAGCTAAAGATACTACTGAAGCCTTTGAAAAAATGGTTTCACTACTTTCT
GTTTTGCTTTCCATGCAGGGTGCTGTAGACATAAACAAGCTTTGTGAAGAAATGCTGGACAACAGGGCAACCTTAC
AAGCTATAGCCTCAGAGTTTAGTTCCCTTCCATCATATGCAGCTTTTGCTACTGCTCAAGAAGCTTATGAGCAGGC
TGTTGCTAATGGTGATTCTGAAGTTGTTCTTAAAAAGTTGAAGAAGTCTTTGAATGTGGCTAAATCTGAATTTGAC
CGTGATGCAGCCATGCAACGTAAGTTGGAAAAGATGGCTGATCAAGCTATGACCCAAATGTATAAACAGGCTAGAT
CTGAGGACAAGAGGGCAAAAGTTACTAGTGCTATGCAGACAATGCTTTTCACTATGCTTAGAAAGTTGGATAATGA
TGCACTCAACAACATTATCAACAATGCAAGAGATGGTTGTGTTCCCTTGAACATAATACCTCTTACAACAGCAGCC
AAACTAATGGTTGTCATACCAGACTATAACACATATAAAAATACGTGTGATGGTACAACATTTACTTATGCATCAG
CATTGTGGGAAATCCAACAGGTTGTAGATGCAGATAGTAAAATTGTTCAACTTAGTGAAATTAGTATGGACAATTC
ACCTAATTTAGCATGGCCTCTTATTGTAACAGCTTTAAGGGCCAATTCTGCTGTCAAATTACAGAATAATGAGCTT
AGTCCTGTTGCACTACGACAGATGTCTTGTGCTGCCGGTACTACACAAACTGCTTGCACTGATGACAATGCGTTAG
CTTACTACAACACAACAAAGGGAGGTAGGTTTGTACTTGCACTGTTATCCGATTTACAGGATTTGAAATGGGCTAG
ATTCCCTAAGAGTGATGGAACTGGTACTATCTATACAGAACTGGAACCACCTTGTAGGTTTGTTACAGACACACCT
AAAGGTCCTAAAGTGAAGTATTTATACTTTATTAAAGGATTAAACAACCTAAATAGAGGTATGGTACTTGGTAGTT
TAGCTGCCACAGTACGTCTACAAGCTGGTAATGCAACAGAAGTGCCTGCCAATTCAACTGTATTATCTTTCTGTGC
TTTTGCTGTAGATGCTGCTAAAGCTTACAAAGATTATCTAGCTAGTGGGGGACAACCAATCACTAATTGTGTTAAG
ATGTTGTGTACACACACTGGTACTGGTCAGGCAATAACAGTTACACCGGAAGCCAATATGGATCAAGAATCCTTTG
GTGGTGCATCGTGTTGTCTGTACTGCCGTTGCCACATAGATCATCCAAATCCTAAAGGATTTTGTGACTTAAAAGG
TAAGTATGTACAAATACCTACAACTTGTGCTAATGACCCTGTGGGTTTTACACTTAAAAACACAGTCTGTACCGTC
TGCGGTATGTGGAAAGGTTATGGCTGTAGTTGTGATCAACTCCGCGAACCCATGCTTCAGTCAGCTGATGCACAAT
CGTTTTTAAACGGGTTTGCGGTGTAAGTGCAGCCCGTCTTACACCGTGCGGCACAGGCACTAGTACTGATGTCGTA
TACAGGGCTTTTGACATCTACAATGATAAAGTAGCTGGTTTTGCTAAATTCCTAAAAACTAATTGTTGTCGCTTCC
AAGAAAAGGACGAAGATGACAATTTAATTGATTCTTACTTTGTAGTTAAGAGACACACTTTCTCTAACTACCAACA
TGAAGAAACAATTTATAATTTACTTAAGGATTGTCCAGCTGTTGCTAAACATGACTTCTTTAAGTTTAGAATAGAC
GGTGACATGGTACCACATATATCACGTCAACGTCTTACTAAATACACAATGGCAGACCTCGTCTATGCTTTAAGGC
ATTTTGATGAAGGTAATTGTGACACATTAAAAGAAATACTTGTCACATACAATTGTTGTGATGATGATTATTTCAA
TAAAAAGGACTGGTATGATTTTGTAGAAAACCCAGATATATTACGCGTATACGCCAACTTAGGTGAACGTGTACGC
CAAGCTTTGTTAAAAACAGTACAATTCTGTGATGCCATGCGAAATGCTGGTATTGTTGGTGTACTGACATTAGATA
ATCAAGATCTCAATGGTAACTGGTATGATTTCGGTGATTTCATACAAACCACGCCAGGTAGTGGAGTTCCTGTTGT
AGATTCTTATTATTCATTGTTAATGCCTATATTAACCTTGACCAGGGCTTTAACTGCAGAGTCACATGTTGACACT
GACTTAACAAAGCCTTACATTAAGTGGGATTTGTTAAAATATGACTTCACGGAAGAGAGGTTAAAACTCTTTGACC
GTTATTTTAAATATTGGGATCAGACATACCACCCAAATTGTGTTAACTGTTTGGATGACAGATGCATTCTGCATTG
TGCAAACTTTAATGTTTTATTCTCTACAGTGTTCCCACCTACAAGTTTTGGACCACTAGTGAGAAAAATATTTGTT
GATGGTGTTCCATTTGTAGTTTCAACTGGATACCACTTCAGAGAGCTAGGTGTTGTACATAATCAGGATGTAAACT
TACATAGCTCTAGACTTAGTTTTAAGGAATTACTTGTGTATGCTGCTGACCCTGCTATGCACGCTGCTTCTGGTAA
TCTATTACTAGATAAACGCACTACGTGCTTTTCAGTAGCTGCACTTACTAACAATGTTGCTTTTCAAACTGTCAAA
CCCGGTAATTTTAACAAAGACTTCTATGACTTTGCTGTGTCTAAGGGTTTCTTTAAGGAAGGAAGTTCTGTTGAAT
TAAAACACTTCTTCTTTGCTCAGGATGGTAATGCTGCTATCAGCGATTATGACTACTATCGTTATAATCTACCAAC
AATGTGTGATATCAGACAACTACTATTTGTAGTTGAAGTTGTTGATAAGTACTTTGATTGTTACGATGGTGGCTGT
ATTAATGCTAACCAAGTCATCGTCAACAACCTAGACAAATCAGCTGGTTTTCCATTTAATAAATGGGGTAAGGCTA
GACTTTATTATGATTCAATGAGTTATGAGGATCAAGATGCACTTTTCGCATATACAAAACGTAATGTCATCCCTAC
TATAACTCAAATGAATCTTAAGTATGCCATTAGTGCAAAGAATAGAGCTCGCACCGTAGCTGGTGTCTCTATCTGT
AGTACTATGACCAATAGACAGTTTCATCAAAAATTATTGAAATCAATAGCCGCCACTAGAGGAGCTACTGTAGTAA
TTGGAACAAGCAAATTCTATGGTGGTTGGCACAACATGTTAAAAACTGTTTATAGTGATGTAGAAAACCCTCACCT
TATGGGTTGGGATTATCCTAAATGTGATAGAGCCATGCCTAACATGCTTAGAATTATGGCCTCACTTGTTCTTGCT
CGCAAACATACAACGTGTTGTAGCTTGTCACACCGTTTCTATAGATTAGCTAATGAGTGTGCTCAAGTATTGAGTG
AAATGGTCATGTGTGGCGGTTCACTATATGTTAAACCAGGTGGAACCTCATCAGGAGATGCCACAACTGCTTATGC
TAATAGTGTTTTTAACATTTGTCAAGCTGTCACGGCCAATGTTAATGCACTTTTATCTACTGATGGTAACAAAATT
GCCGATAAGTATGTCCGCAATTTACAACACAGACTTTATGAGTGTCTCTATAGAAATAGAGATGTTGACACAGACT
TTGTGAATGAGTTTTACGCATATTTGCGTAAACATTTCTCAATGATGATACTCTCTGACGATGCTGTTGTGTGTTT
CAATAGCACTTATGCATCTCAAGGTCTAGTGGCTAGCATAAAGAACTTTAAGTCAGTTCTTTATTATCAAAACAAT
GTTTTTATGTCTGAAGCAAAATGTTGGACTGAGACTGACCTTACTAAAGGACCTCATGAATTTTGCTCTCAACATA
CAATGCTAGTTAAACAGGGTGATGATTATGTGTACCTTCCTTACCCAGATCCATCAAGAATCCTAGGGGCCGGCTG
TTTTGTAGATGATATCGTAAAAACAGATGGTACACTTATGATTGAACGGTTCGTGTCTTTAGCTATAGATGCTTAC
CCACTTACTAAACATCCTAATCAGGAGTATGCTGATGTCTTTCATTTGTACTTACAATACATAAGAAAGCTACATG
ATGAGTTAACAGGACACATGTTAGACATGTATTCTGTTATGCTTACTAATGATAACACTTCAAGGTATTGGGAACC
TGAGTTTTATGAGGCTATGTACACACCGCATACAGTCTTACAGGCTGTTGGGGCTTGTGTTCTTTGCAATTCACAG
ACTTCATTAAGATGTGGTGCTTGCATACGTAGACCATTCTTATGTTGTAAATGCTGTTACGACCATGTCATATCAA
CATCACATAAATTAGTCTTGTCTGTTAATCCGTATGTTTGCAATGCTCCAGGTTGTGATGTCACAGATGTGACTCA
ACTTTACTTAGGAGGTATGAGCTATTATTGTAAATCACATAAACCACCCATTAGTTTTCCATTGTGTGCTAATGGA
CAAGTTTTTGGTTTATATAAAAATACATGTGTTGGTAGCGATAATGTTACTGACTTTAATGCAATTGCAACATGTG
ACTGGACAAATGCTGGTGATTACATTTTAGCTAACACCTGTACTGAAAGACTCAAGCTTTTTGCAGCAGAAACGCT
CAAAGCTACTGAGGAGACATTTAAACTGTCTTATGGTATTGCTACTGTACGTGAAGTGCTGTCTGACAGAGAATTA
CATCTTTCATGGGAAGTTGGTAAACCTAGACCACCACTTAACCGAAATTATGTCTTTACTGGTTATCGTGTAACTA
AAAACAGTAAAGTACAAATAGGAGAGTACACCTTTGAAAAAGGTGACTATGGTGATGCTGTTGTTTACCGAGGTAC
AACAACTTACAAATTAAATGTTGGTGATTATTTTGTGCTGACATCACATACAGTAATGCCATTAAGTGCACCTACA
CTAGTGCCACAAGAGCACTATGTTAGAATTACTGGCTTATACCCAACACTCAATATCTCAGATGAGTTTTCTAGCA
ATGTTGCAAATTATCAAAAGGTTGGTATGCAAAAGTATTCTACACTCCAGGGACCACCTGGTACTGGTAAGAGTCA
TTTTGCTATTGGCCTAGCTCTCTACTACCCTTCTGCTCGCATAGTGTATACAGCTTGCTCTCATGCCGCTGTTGAT
GCACTATGTGAGAAGGCATTAAAATATTTGCCTATAGATAAATGTAGTAGAATTATACCTGCACGTGCTCGTGTAG
AGTGTTTTGATAAATTCAAAGTGAATTCAACATTAGAACAGTATGTCTTTTGTACTGTAAATGCATTGCCTGAGAC
GACAGCAGATATAGTTGTCTTTGATGAAATTTCAATGGCCACAAATTATGATTTGAGTGTTGTCAATGCCAGATTA
CGTGCTAAGCACTATGTGTACATTGGCGACCCTGCTCAATTACCTGCACCACGCACATTGCTAACTAAGGGCACAC
TAGAACCAGAATATTTCAATTCAGTGTGTAGACTTATGAAAACTATAGGTCCAGACATGTTCCTCGGAACTTGTCG
GCGTTGTCCTGCTGAAATTGTTGACACTGTGAGTGCTTTGGTTTATGATAATAAGCTTAAAGCACATAAAGACAAA
TCAGCTCAATGCTTTAAAATGTTTTATAAGGGTGTTATCACGCATGATGTTTCATCTGCAATTAACAGGCCACAAA
TAGGCGTGGTAAGAGAATTCCTTACACGTAACCCTGCTTGGAGAAAAGCTGTCTTTATTTCACCTTATAATTCACA
GAATGCTGTAGCCTCAAAGATTTTGGGACTACCAACTCAAACTGTTGATTCATCACAGGGCTCAGAATATGACTAT
GTCATATTCACTCAAACCACTGAAACAGCTCACTCTTGTAATGTAAACAGATTTAATGTTGCTATTACCAGAGCAA
AAGTAGGCATACTTTGCATAATGTCTGATAGAGACCTTTATGACAAGTTGCAATTTACAAGTCTTGAAATTCCACG
TAGGAATGTGGCAACTTTACAAGCTGAAAATGTAACAGGACTCTTTAAAGATTGTAGTAAGGTAATCACTGGGTTA
CATCCTACACAGGCACCTACACACCTCAGTGTTGACACTAAATTCAAAACTGAAGGTTTATGTGTTGACATACCTG
GCATACCTAAGGACATGACCTATAGAAGACTCATCTCTATGATGGGTTTTAAAATGAATTATCAAGTTAATGGTTA
CCCTAACATGTTTATCACCCGCGAAGAAGCTATAAGACATGTACGTGCATGGATTGGCTTCGATGTCGAGGGGTGT
CATGCTACTAGAGAAGCTGTTGGTACCAATTTACCTTTACAGCTAGGTTTTTCTACAGGTGTTAACCTAGTTGCTG
TACCTACAGGTTATGTTGATACACCTAATAATACAGATTTTTCCAGAGTTAGTGCTAAACCACCGCCTGGAGATCA
ATTTAAACACCTCATACCACTTATGTACAAAGGACTTCCTTGGAATGTAGTGCGTATAAAGATTGTACAAATGTTA
AGTGACACACTTAAAAATCTCTCTGACAGAGTCGTATTTGTCTTATGGGCACATGGCTTTGAGTTGACATCTATGA
AGTATTTTGTGAAAATAGGACCTGAGCGCACCTGTTGTCTATGTGATAGACGTGCCACATGCTTTTCCACTGCTTC
AGACACTTATGCCTGTTGGCATCATTCTATTGGATTTGATTACGTCTATAATCCGTTTATGATTGATGTTCAACAA
TGGGGTTTTACAGGTAACCTACAAAGCAACCATGATCTGTATTGTCAAGTCCATGGTAATGCACATGTAGCTAGTT
GTGATGCAATCATGACTAGGTGTCTAGCTGTCCACGAGTGCTTTGTTAAGCGTGTTGACTGGACTATTGAATATCC
TATAATTGGTGATGAACTGAAGATTAATGCGGCTTGTAGAAAGGTTCAACACATGGTTGTTAAAGCTGCATTATTA
GCAGACAAATTCCCAGTTCTTCACGACATTGGTAACCCTAAAGCTATTAAGTGTGTACCTCAAGCTGATGTAGAAT
GGAAGTTCTATGATGCACAGCCTTGTAGTGACAAAGCTTATAAAATAGAAGAATTATTCTATTCTTATGCCACACA
TTCTGACAAATTCACAGATGGTGTATGCCTATTTTGGAATTGCAATGTCGATAGATATCCTGCTAATTCCATTGTT
TGTAGATTTGACACTAGAGTGCTATCTAACCTTAACTTGCCTGGTTGTGATGGTGGCAGTTTGTATGTAAATAAAC
ATGCATTCCACACACCAGCTTTTGATAAAAGTGCTTTTGTTAATTTAAAACAATTACCATTTTTCTATTACTCTGA
CAGTCCATGTGAGTCTCATGGAAAACAAGTAGTGTCAGATATAGATTATGTACCACTAAAGTCTGCTACGTGTATA
ACACGTTGCAATTTAGGTGGTGCTGTCTGTAGACATCATGCTAATGAGTACAGATTGTATCTCGATGCTTATAACA
TGATGATCTCAGCTGGCTTTAGCTTGTGGGTTTACAAACAATTTGATACTTATAACCTCTGGAACACTTTTACAAG
ACTTCAGAGTTTAGAAAATGTGGCTTTTAATGTTGTAAATAAGGGACACTTTGATGGACAACAGGGTGAAGTACCA
GTTTCTATCATTAATAACACTGTTTACACAAAAGTTGATGGTGTTGATGTAGAATTGTTTGAAAATAAAACAACAT
TACCTGTTAATGTAGCATTTGAGCTTTGGGCTAAGCGCAACATTAAACCAGTACCAGAGGTGAAAATACTCAATAA
TTTGGGTGTGGACATTGCTGCTAATACTGTGATCTGGGACTACAAAAGAGATGCTCCAGCACATATATCTACTATT
GGTGTTTGTTCTATGACTGACATAGCCAAGAAACCAACTGAAACGATTTGTGCACCACTCACTGTCTTTTTTGATG
GTAGAGTTGATGGTCAAGTAGACTTATTTAGAAATGCCCGTAATGGTGTTCTTATTACAGAAGGTAGTGTTAAAGG
TTTACAACCATCTGTAGGTCCCAAACAAGCTAGTCTTAATGGAGTCACATTAATTGGAGAAGCCGTAAAAACACAG
TTCAATTATTATAAGAAAGTTGATGGTGTTGTCCAACAATTACCTGAAACTTACTTTACTCAGAGTAGAAATTTAC
AAGAATTTAAACCCAGGAGTCAAATGGAAATTGATTTCTTAGAATTAGCTATGGATGAATTCATTGAACGGTATAA
ATTAGAAGGCTATGCCTTCGAACATATCGTTTATGGAGATTTTAGTCATAGTCAGTTAGGTGGTTTACATCTACTG
ATTGGACTAGCTAAACGTTTTAAGGAATCACCTTTTGAATTAGAAGATTTTATTCCTATGGACAGTACAGTTAAAA
ACTATTTCATAACAGATGCGCAAACAGGTTCATCTAAGTGTGTGTGTTCTGTTATTGATTTATTACTTGATGATTT
TGTTGAAATAATAAAATCCCAAGATTTATCTGTAGTTTCTAAGGTTGTCAAAGTGACTATTGACTATACAGAAATT
TCATTTATGCTTTGGTGTAAAGATGGCCATGTAGAAACATTTTACCCAAAATTACAATCTAGTCAAGCGTGGCAAC
CGGGTGTTGCTATGCCTAATCTTTACAAAATGCAAAGAATGCTATTAGAAAAGTGTGACCTTCAAAATTATGGTGA
TAGTGCAACATTACCTAAAGGCATAATGATGAATGTCGCAAAATATACTCAACTGTGTCAATATTTAAACACATTA
ACATTAGCTGTACCCTATAATATGAGAGTTATACATTTTGGTGCTGGTTCTGATAAAGGAGTTGCACCAGGTACAG
CTGTTTTAAGACAGTGGTTGCCTACGGGTACGCTGCTTGTCGATTCAGATCTTAATGACTTTGTCTCTGATGCAGA
TTCAACTTTGATTGGTGATTGTGCAACTGTACATACAGCTAATAAATGGGATCTCATTATTAGTGATATGTACGAC
CCTAAGACTAAAAATGTTACAAAAGAAAATGACTCTAAAGAGGGTTTTTTCACTTACATTTGTGGGTTTATACAAC
AAAAGCTAGCTCTTGGAGGTTCCGTGGCTATAAAGATAACAGAACATTCTTGGAATGCTGATCTTTATAAGCTCAT
GGGACACTTCGCATGGTGGACAGCCTTTGTTACTAATGTGAATGCGTCATCATCTGAAGCATTTTTAATTGGATGT
AATTATCTTGGCAAACCACGCGAACAAATAGATGGTTATGTCATGCATGCAAATTACATATTTTGGAGGAATACAA
ATCCAATTCAGTTGTCTTCCTATTCTTTATTTGACATGAGTAAATTTCCCCTTAAATTAAGGGGTACTGCTGTTAT
GTCTTTAAAAGAAGGTCAAATCAATGATATGATTTTATCTCTTCTTAGTAAAGGTAGACTTATAATTAGAGAAAAC
AACAGAGTTGTTATTTCTAGTGATGTTCTTGTTAACAACTAAGCGGCCGCACGAACAATGGTTTCTAAGGGAGAAG
AACTCTTTACTGGTGTTGTCCCAATTCTGGTTGAGCTGGATGGTGATGTGAATGGCCACAAATTCTCTGTGTCTGG
TGAAGGTGAAGGAGATGCAACTTATGGAAAGCTGACTCTGAAGTTCATTTGTACAACAGGAAAGCTGCCAGTGCCT
TGGCCAACTCTGGTGACCACCCTGACTTATGGTGTTCAATGTTTCAGCAGGTACCCTGACCACATGAAGCAGCATG
ACTTCTTTAAATCTGCAATGCCAGAAGGTTATGTTCAGGAGAGGACAATCTTCTTTAAGGATGATGGAAATTATAA
GACAAGGGCAGAAGTGAAGTTTGAAGGTGATACACTGGTTAACAGAATTGAGCTGAAAGGCATTGATTTTAAGGAA
GATGGAAACATTCTGGGTCACAAGCTGGAGTACAACTATAATTCTCACAATGTTTACATTATGGCAGATAAGCAGA
GGAATGGAATTAAGGCTAATTTCAAGATTAGACACAACATTGAGGATGGATCTGTCCAACTGGCAGACCATTACCA
GCAGAACACCCCTATTGGTGATGGCCCAGTTCTCCTCCCAGATAATCACTATCTCAGCACTCAATCTGCTCTGTCC
AAAGACCCTAATGAGAAAAGAGACCACATGGTCCTCCTGGAGTTTGTGACAGCAGCAGGAATTACTCTGGGAATGG
ATGAGCTGTACAAGGGTAAGTCACTGACTGTCTATGCCTGGGAAAGGGTGGGCAGGAGATGGGGCAGTGCAGGAAA
AGTGGCACTATGAACCCACTAGTTTGACAATTAATCATAAGCATAGTATAATACAACTCACTATAGCAATTGTACT
AACCTTCTTCTCTTTCCTCTCCTGACAGGAGGAGCCATCATGAAGACCTTCAACATCTCTCAGCAGGACCTTGAGC
TGGTGGAAGTTGCCACTGAGAAAATCACCATGCTCTATGAGGACAACAAGCACCATGTTGGTGCTGCCATCAGGAC
CAAGACAGGAGAAATCATCTCTGCTGTCCACATTGAAGCCTACATTGGCAGGGTCACTGTGTGTGCAGAGGCCATT
GCCATTGGGTCTGCAGTCTCCAATGGGCAGAAGGACTTTGACACCATTGTGGCTGTGAGGCACCCCTACAGTGATG
AGGTGGACAGGTCCATCAGAGTGGTGTCCCCCTGTGGCATGTGCAGGGAGCTCATCTCAGACTATGCCCCTGATTG
CTTTGTTCTGATTGAGATGAATGGCAAGCTGGTCAAGACAACCATTGAGGAGCTGATTCCACTGAAATACACCAGA
AACTAAACGAACAAACTAAAATGTCTGATAATGGACCCCAAAATCAGCGAAATGCACCCCGCATTACGTTTGGTGG
ACCCTCAGATTCAACTGGCAGTAACCAGAATGGAGAACGCAGTGGGGCGCGATCAAAACAACGTCGGCCCCAAGGT
TTACCCAATAATACTGCGTCTTGGTTCACCGCTCTCACTCAACATGGCAAGGAAGACCTTAAATTCCCTCGAGGAC
AAGGCGTTCCAATTAACACCAATAGCAGTCCAGATGACCAAATTGGCTACTACCGAAGAGCTACCAGACGAATTCG
TGGTGGTGACGGTAAAATGAAAGATCTCAGTCCAAGATGGTATTTCTACTACCTAGGAACTGGGCCAGAAGCTGGA
CTTCCCTATGGTGCTAACAAAGACGGCATCATATGGGTTGCAACTGAGGGAGCCTTGAATACACCAAAAGATCACA
TTGGCACCCGCAATCCTGCTAACAATGCTGCAATCGTGCTACAACTTCCTCAAGGAACAACATTGCCAAAAGGCTT
CTACGCAGAAGGGAGCAGAGGCGGCAGTCAAGCCTCTTCTCGTTCCTCATCACGTAGTCGCAACAGTTCAAGAAAT
TCAACTCCAGGCAGCAGTAGGGGAACTTCTCCTGCTAGAATGGCTGGCAATGGCGGTGATGCTGCTCTTGCTTTGC
TGCTGCTTGACAGATTGAACCAGCTTGAGAGCAAAATGTCTGGTAAAGGCCAACAACAACAAGGCCAAACTGTCAC
TAAGAAATCTGCTGCTGAGGCTTCTAAGAAGCCTCGGCAAAAACGTACTGCCACTAAAGCATACAATGTAACACAA
GCTTTCGGCAGACGTGGTCCAGAACAAACCCAAGGAAATTTTGGGGACCAGGAACTAATCAGACAAGGAACTGATT
ACAAACATTGGCCGCAAATTGCACAATTTGCCCCCAGCGCTTCAGCGTTCTTCGGAATGTCGCGCATTGGCATGGA
AGTCACACCTTCGGGAACGTGGTTGACCTACACAGGTGCCATCAAATTGGATGACAAAGATCCAAATTTCAAAGAT
CAAGTCATTTTGCTGAATAAGCATATTGACGCATACAAAACATTCCCACCAACAGAGCCTAAAAAGGACAAAAAGA
AGAAGGCTGATGAAACTCAAGCCTTACCGCAGAGACAGAAGAAACAGCAAACTGTGACTCTTCTTCCTGCTGCAGA
TTTGGATGATTTCTCCAAACAATTGCAACAATCCATGAGCAGTGCTGACTCAACTCAGGCCTAAACTCATGCAGAC
CACACAAGGCAGATGGGCTATATAAACGTTTTCGCTTTTCCGTTTACGATATATAGTCTACTCTTGTGCAGAATGA
ATTCTCGTAACTACATAGCACAAGTAGATGTAGTTAACTTTAATCTCACATAGCAATCTTTAATCAGTGTGTAACA
TTAGGGAGGACTTGAAAGAGCCACCACATTTTCACCGAGGCCACGCGGAGTACGATCGAGTGTACAGTGAACAATG
CTAGGGAGAGCTGCCTATATGGAAGAGCCCTAATGTGTAAAATTAATTTTAGTAGTGCTATCCCCATGTGATTTTA
ATAGCTTCTTAGGAGAATGACAAAAAAAAAAAAAAAAAAAAAAAAAAAAAAAAAGGGCGGCATGGTCCCAGCCTCC
TCGCTGGCGCCGCCTGGGCAACATGCTTCGGCATGGCGAATGGGACCGGATCCGGGGGCGCGCCTGTGCCTTCTAG
TTGCCAGCCATCTGTTGTTTGCCCCTCCCCCGTGCCTTCCTTGACCCTGGAAGGTGCCACTCCCACTGTCCTTTCC
TAATAAAATGAGGAAATTGCATCGCATTGTCTGAGTAGGTGTCATTCTATTCTGGGGGGTGGGGTGGGGCAGGACA
GCAAGGGGGAGGATTGGGAAGACAATAGCAGGCATGCTGGGGATGCGGTGGGCTCTATGG

Hammerhead virus ribozyme site (HHV Rz) and Hepatitis Delta virus ribozyme site (HDV Rz) were inserted immediately before 5′ end and after the 3′ end of SARS2, respectively, to allow removal of extra nucleotides at both 5′ and 3′ ends from the nascent RNA transcribed from the replicon DNA either by cellular transcription machinery or the RNA from in vitro transcription, so that the RNA replicon with authentic 5′ and 3′ ends were produced to faithfully recapitulate RNA replication and transcription. For the same reason, NSP1 and ORF10 adjacent to the 5′ end and 3′ end were kept in the replicon design. HDV Rz would also allow direct use of the DNA replicon for in vitro transcription without linearizing. The firefly luciferase reporter gene (fLuc) was inserted between NSP1 and NSP2-16 to monitor translation and replication/transcription of the replicon, while the GFP::Bsr fusion gene was inserted between NSP16 and nucleocapsid N gene to monitor replication/transcription of the replicon and selection of stable cell replicons. The nucleocapsid N gene and its transcriptional regulatory sequence (TRS) were kept for efficient SARS gRNA and N sgRNA replication and N protein expression [40]. The TRS of the S gene was inserted before the GFP::Bsr fusion gene for GFP::Bsr sgRNA replication and GFP::Bsr protein expression. Porcine teschovirus-1 self-cleaving peptide 2A (P2A) was inserted between NSP1 and fLuc to ensure proper processing of NSP1 and fLuc. Encephalomarcarditis virus internal ribozyme entry site (IRES) was inserted before the NSP2-16 gene to facilitate translation of the large polypeptide NSP2-16 from the RNA replicon. Bovine growth hormone polyadenylation signal (BGH pA) was added to the a end to stabilize the RNA.

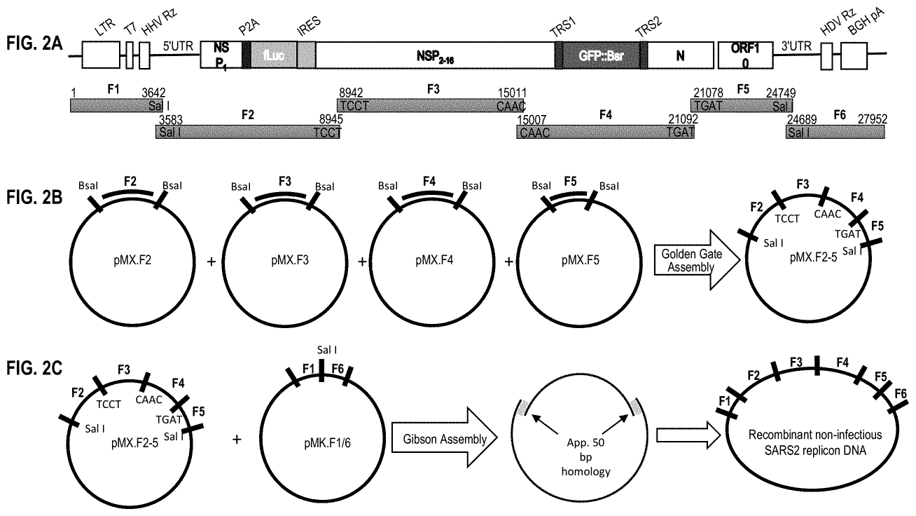

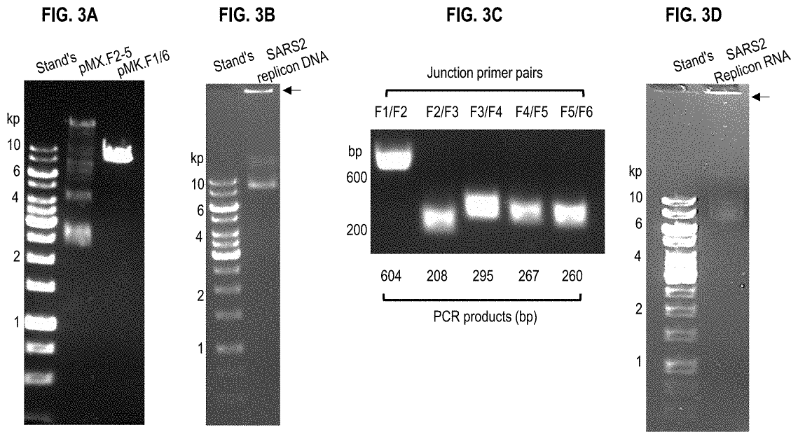

To construct the two-in-one dual reporter SARS2 DNA replicon construct (27,952 nucleotides), the replicon was divided to 5 fragments (F1/6 and F2, F3, F4, and F5, FIG. 2A) and synthesized onto the pMX vector (for F2-5) or pMK vector (for F1/6) with appropriate restriction sites (BasI or SalI) for subsequently cloning and validated by sequencing. F2-5 were first ligated using a Golden Gate Assembly kit (FIG. 2B). The intermediate construct pMX.F2-5 was digested with SalI and annealed with SalI-digested pMK.F1/6 using a Gibson Assembly kit (FIG. 2C, FIG. 3A) to obtain the final DNA construct pMK.F1-6, the DNA replicon (FIG. 3B). The DNA replicon was verified by PCR with 5 pairs of primers spanning each of the five adjacent junctions, which (FIG. 3C). To synthesize the RNA replicon, the DNA replicon was used as the template in an in vitro transcription with inclusion of Ribo m7G Cap analog. The capped RNA replicon was purified, analyzed by denatured agarose gel electrophoresis, and estimated to be the right size (FIG. 3D).

### Example 2: Expression of the Reporter Genes and SARS2 N Gene from the LTR/T7 Dual Promoter-Driven and GFP/fLuc Dual Reporter-Expressing SARS2 Replicon

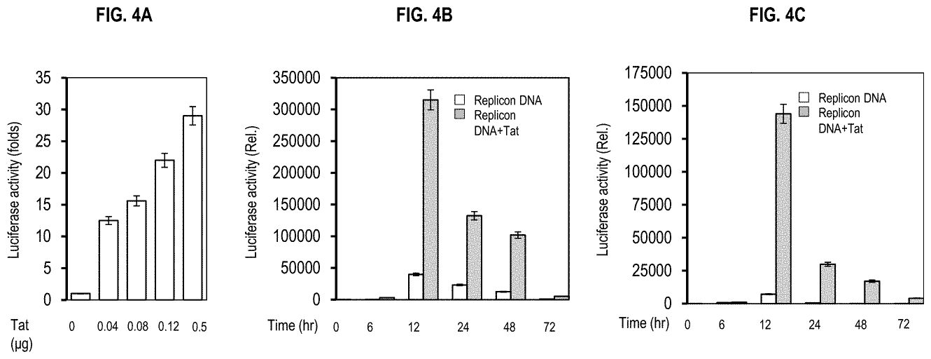

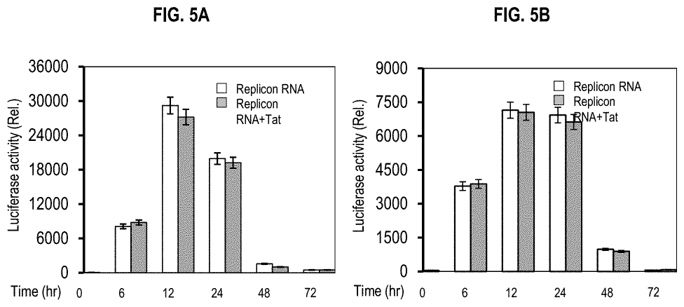

Gene expression from the new replicon was tested. The first reporter gene fLuc was inserted between NSP1 and NSP2-16. So the fLuc expression could be used as an indicator of translation of positive-stranded RNA that was either transcribed from the replicon DNA, the in vitro transcribed replicon RNA, or gRNA resulting from replication of these initial transcribed RNA. First, the luciferase (fLuc) expression in cells transfected with the replicon construct was tested and effects of HIV Tat expression on the fLuc expression. fLuc expression, measured by the fLuc activity, was detected in 293T transfected with the replicon DNA alone and increased in 293T co-transfected with Tat expression plasmid pc3.Tat in a dose-dependent manner (FIG. 4A). fLuc expression showed increases up to 12 hours and then decreased to the minimal level at 72 hours in 293T transfected with the replicon DNA alone, while fLuc expression had a similar kinetics but at a much higher level at each time point in cells co-transfected with pc3.Tat (FIG. 4B). Similar results were obtained in Vero6 (FIG. 4C). fLuc expression was next assayed in cells transfected with the in vitro transcribed replicon RNA. fLuc expression showed generally similar kinetics in 293T transfected with the replicon RNA (FIG. 5A) and Vero6 transfected with the replicon RNA (FIG. 5B). Tat co-transfection did not show any effects on fLuc expression in cells transfected with the replicon RNA. Compared to the replicon DNA transfection (FIGS. 4B & 4C), the replicon RNA transfection gave rise to a higher level of fLuc expression at 6 hours post transfection and had only relatively slight decreases at 24 hours from 12 hours (FIGS. 5A & 5B, 5C).

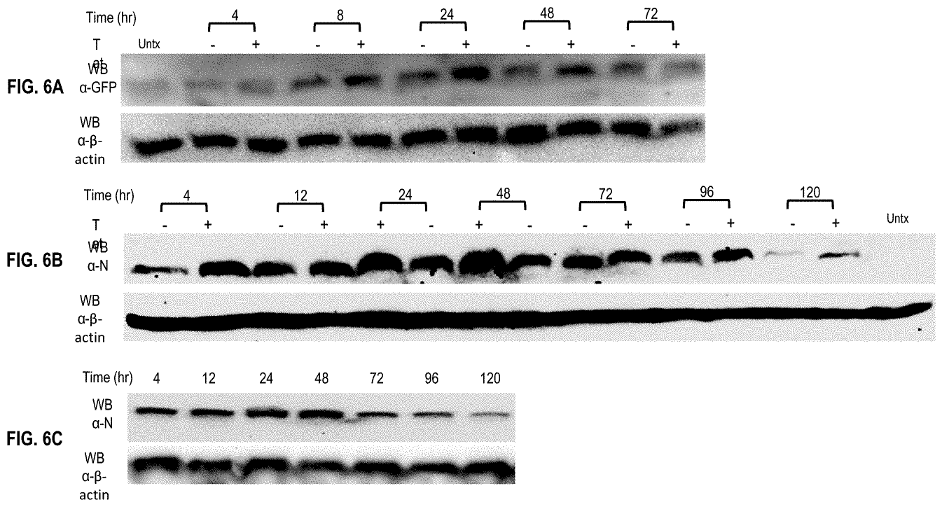

The second reporter gene GFP was inserted as a fusion protein with Blasticidin (Bsr) between NSP2-16 and N gene, and the GFP gene was preceded with the authentic transcriptional regulatory sequences (TRS) of SARS2 S gene. Thus, GFP expression would represent replication of gRNA and GFP sgRNA and translation of GFP sgRNA. GFP expression was detected in 293T transfected with the replicon DNA at 4 hours post transfection, increased up to 48 hours and then decreased (FIG. 6A). Tat co-transfection led to a higher level of GFP expression at each time point of the detection. There was only very dim GFP expression in these cells observed under a fluorescence microscope (data not shown). The only SARS2 structural gene nucleocapsid N and its native TRS configuration were kept in the design of the replicon, as it has been to be required for formation of the replication/transcription complex and replication of coronaviruses [40]. N protein expression would provide additional evidence to support replication of gRNA and sgRNA (N) and translation of N sgRNA. N protein showed similar expression kinetics and response to Tat co-transfection in to those of GFP expression (FIG. 6B). The N protein was also detected in a similar kinetics in 293T transfected with the replicon RNA (FIG. 6C). Similarly, GFP and N proteins were detected in Vero6 transfected with the replicon DNA, DNA plus Tat, or the replicon RNA by Western blotting except for that GFP expression was readily observed in Vero6 under a fluorescence microscope (data not shown).

### Example 3: RNA Transcription and Replication from the LTR/T7 Dual Promoter-Driven and GFP/fLuc Dual Reporter-Expressing SARS2 Replicon and its Inhibition by Remdesivir

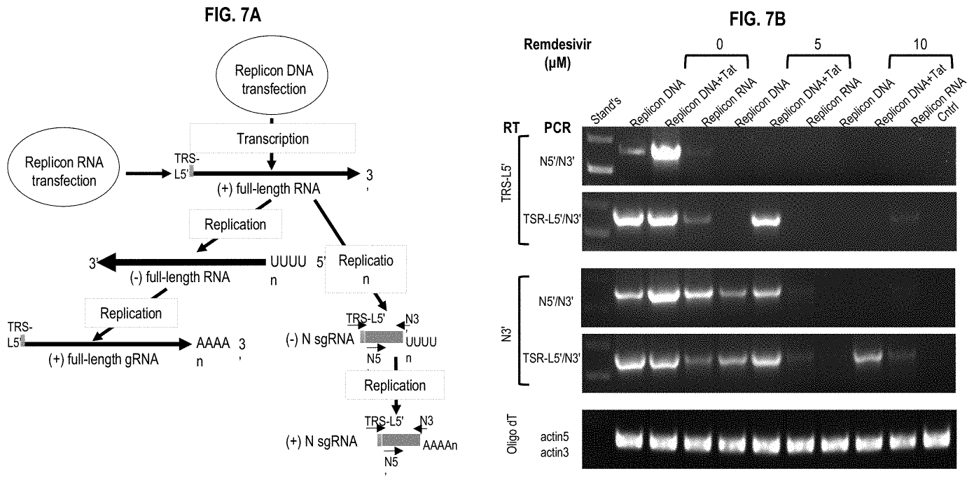

The full-length SARS2 replicon RNA could be derived from in vitro transcription of the replicon DNA or transcription of the replicon DNA in cells by cellular transcription machinery, and serve as the template for translation and expression of NSP1-16 and for synthesis of negative-stranded gRNA and sgRNA intermediates, which in turn served as the templates for subsequent synthesis positive-stranded gRNA and sgRNA (FIG. 7A). Thus, detection of negative-stranded RNA gRNA and sgRNA were used as an indicator for the replicon replication. An oligonucleotide TRS-L5 aligned to the TRS leader that was present in the full-length gRNA and sgRNA was used as the RT primer to synthesize the cDNA [26, 48-52], followed by PCR using primers TRS-L5/N3′ for detection of negative-stranded gRNA, or PCR using primers N5′/N3′ for detection of negative-stranded N sgRNA. Total RNA was isolated from 293T transfected with the replicon DNA alone, the replicon DNA and Tat, or the replicon RNA, and then treated with RNase-Free DNase, followed by multiple rounds of acidic phenol extraction to eliminate input DNA contamination, which was verified by the absence of PCR products using the RNA samples and the replicon DNA-specific primers (FIG. 3C) as the template (data not shown). RT by TRS-L5′, followed by PCR with N5′/N3′ and TRS-L5/N3 showed expression of negative-stranded N sgRNA and gRNA in all transfections (upper two panels, FIG. 7B). In the meantime, RT was performed using N3′ as the primer, followed by PCR also showed expression of positive-stranded N sgRNA and gRNA in all the transfections (middle two panels, FIG. 7B). Importantly, Remdesivir, a nucleoside analog and an RNA-dependent RNA polymerase inhibitor that has been used for treatment of COVID-19 patients [53-57], showed inhibition at a concentration of 5 μM and even greater inhibition at a concentration of 10 μM of positive- and negative-stranded gRNA and N sgRNA expression.

### Example 4: Inhibition of Gene Expression from the LTR/T7 Dual Promoter-Driven and GFP/fLuc Dual Reporter-Expressing SARS2 Replicon by Remdesivir

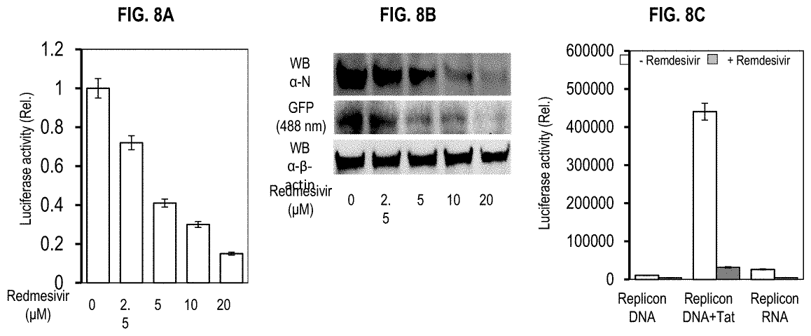

The effects of Remdesivir was tested on expression of these two reporter genes and SARS nucleocapsid N gene from the replicon. 293T were transfected with the replicon and treated with different concentrations of Remdesivir. Remdesivir inhibited the fLuc gene expression in a concentration-dependent manner (FIG. 8A). Expression of GFP and N genes showed similar kinetics of inhibition by Remdesivir (FIG. 8B). Compared to the DNA replicon in which Remdesivir (10 μM) inhibited fLuc by 70%, the same concentration of Remdesivir inhibited fLuc from the DNA replicon and Tat by about 15-fold and from the RNA replicon by about 18-fold (FIG. 8C).

### Discussion

This disclosure provides the engineering, construction and characterization of a dual promoter-driven and dual reporter-expressing SARS2 replicon. This replicon construct contained the genomic organization from 5′ end to 3′ end: LTR-T7-HHD Rz-5′UTR-NSP1-P2A-fLuc-IRES-NSP2-16-TRS-GFP::Bsr-TRS-N-ORF10-3′UTR-HDV Rz-BGH pA. Over the past two years, several SARS-CoV-2 replicons have been developed [58-67]. The general genomic composition of these SARS-CoV-2 replicons includes 5′UTR, ORF1a/1b, a Luc gene or green fluorescence protein (GFP) reporter gene, N, and 3′UTR from 5′ end to 3′ end. But they differ in how the replicon RNA is produced. Some replicons have a T7 promoter at the 5′ end, the replicon RNA has to be synthesized in vitro [58-63]. Other replicons have the human cytomegalovirus (CMV) immediate-early enhance and promoter at the 5′ end, the replicon RNA is transcribed from the replicon DNA that is introduced into cells by transfection [64-67]. There were several main features that still remained unique to the replicon as disclosed herein. These included efficient full-length RNA transcription under the HIV LTR promoter and its transactivation by Tat co-expression, production of the replicon RNA with 5′ and 3′ ends that are identical to the native SARS2, fLuc insertion within the NSP genes as the indicator for translation and replication/transcription of the replicon RNA, placement of IRES before NSP2-16 to facilitate NSP2-16 translation and expression, and one cassette 5UTR-NSP1 at the 5′ end and one cassette ORF10-3′UTR at the 3′ end to maintain the native RNA secondary structure for RNA translation, replication and transcription.

The replicon construct presented herein, when transfected into cells in the form of DNA, or RNA that was transcribed from the DNA, showed successful expression of reporter genes fLuc, GFP, and SARS2 structural gene N in 293T and Vero6. The construct also demonstrated expression of negative-stranded gRNA and SARS2 N sgRNA, which provided additional evidence to support expression and replication of the replicon in these cells. Lastly, the construct demonstrated that an RNA-dependent RNA polymerase inhibitor that has been used to treat SARS2 infection inhibited expression of reporter genes fLuc, GFP, and SARS2 structural gene N and negative- and positive-stranded gRNA and SARS2 N sgRNA in 293T and Vero6. Two other cell lines Hela and Huh7 were also tested in this study and obtained similar results (data not shown). All the findings together support the notion that the new replicon could be used as a surrogate system for screening and identifying anti-SARS2 antiviral drugs and for studying the molecular mechanisms of the host and viral control of SARS2 replication and transcription.

The reporter gene GFP was introduced as an indicator to monitor RNA replication/transcription from the replicon. Surprisingly, only GFP expression was detected on Western blots and only very weak signal under the fluorescence microscope. This phenomenon did appear to be cell type-dependent, as brighter GFP was detected in Vero6 than 293T. This may be due to conformational changes of GFP in the GFP-Bsr fusion protein. Establishing stable cells comprising the replicon construct was attempted, by single cell or bulking cloning with inclusion of Bsr in the culture medium and passages, using different cell lines, and using both the replicon DNA transfection and the replicon RNA transfection; however, attempts were unsuccessful. Thus, these studies were performed using the transient replicon by transfection. Nevertheless, of note is that among all 10 published SARS2 replicons so far, only two SARS2 replicons lead to creation of stable replicon cells [58, 67], and the other eight all appear to be transient replicons [59-66]. Further understanding of the design and organizational differences between these two groups of SARS2 replicons may lead to identification of viral and host factors necessary for SARS2 replication as well as help construct better SARS2 replicons for anti-SARS2 drug screening and evaluation.

Having described the subject matter of the disclosure in detail and by reference to specific embodiments thereof, it will be apparent that modifications and variations are possible without departing from the scope of the claimed subject matter. More specifically, although some aspects of the present disclosure are identified herein as particularly advantageous, it is contemplated that the present subject matter is not necessarily limited to these particular aspects of the claimed subject matter.

### REFERENCES

1. Zhang, G., et al., Analysis of clinical characteristics and laboratory findings of 95 cases of 2019 novel coronavirus pneumonia in Wuhan, China: a retrospective analysis. Respir Res, 2020. 21(1): p. 74.
2. Tan, W, et al., A Novel Coronavirus Genome Identified in a Cluster of Pneumonia Cases—Wuhan, China 2019-2020. China CDC Wkly, 2020. 2(4): p. 61-62.
3. Meng, H., et al., CT imaging and clinical course of asymptomatic cases with COVID-19 pneumonia at admission in Wuhan, China. J Infect, 2020. 81(1): p. e33-e39.
4. Chen, N., et al., Epidemiological and clinical characteristics of 99 cases of 2019 novel coronavirus pneumonia in Wuhan, China: a descriptive study. Lancet, 2020. 395(10223): p. 507-513.
5. WHO. 2022.
6. Zhou, P, et al., A pneumonia outbreak associated with a new coronavirus of probable bat origin. Nature, 2020. 579(7798): p. 270-273.
7. Zhu, N., et al., A Novel Coronavirus from Patients with Pneumonia in China, 2019. N Engl J Med, 2020. 382(8): p. 727-733.
8. Pu, R., et al., The screening value of RT-LAMP and RT-PCR in the diagnosis of COVID-19: systematic review and meta-analysis. J Virol Methods, 2022. 300: p. 114392.
9. Ionescu, M. A., COVID-19 skin lesions are rarely positive at RT-PCR test: the macrophage activation with vascular impact and SARS-CoV-2-induced cytokine storm. Int J Dermatol, 2022. 61(1): p. 3-6.
10. Szabo, G. T, A. J. Mahiny, and I. Vlatkovic, COVID-19 mRNA vaccines: Platforms and current developments. Mol Ther, 2022.
11. Rahman, M. M., et al., A comprehensive review on COVID-19 vaccines: development, effectiveness, adverse effects, distribution and challenges. Virus disease, 2022: p. 1-22.
12. Jensen, A., et al., COVID-19 vaccines: Considering sex differences in efficacy and safety. Contemp Clin Trials, 2022. 115: p. 106700.
13. Huang, Z., et al., A review of the safety and efficacy of current COVID-19 vaccines. Front Med, 2022.
14. Vitiello, A. and F Ferrara, Association and pharmacological synergism of the triple drug therapy baricitinib/remdesivir/rhACE2 for the management of COVID-19 infection. Naunyn Schmiedebergs Arch Pharmacol, 2022. 395(1): p. 99-104.
15. Pagliano, P, et al., An overview of the preclinical discovery and development of remdesivir for the treatment of coronavirus disease 2019 (COVID-19). Expert Opin Drug Discov, 2022. 17(1): p. 9-18.
16. Kaka, A. S., et al., Major Update 2: Remdesivir for Adults With COVID-19: A Living Systematic Review and Meta-analysis for the American College of Physicians Practice Points. Ann Intern Med, 2022.
17. Angamo, M. T, M. A. Mohammed, and G. M. Peterson, Efficacy and safety of remdesivir in hospitalised COVID-19 patients: a systematic review and meta-analysis. Infection, 2022. 50(1): p. 27-41.
18. Pourkarim, F, S. Pourtaghi-Anvarian, and H. Rezaee, Molnupiravir: A new candidate for COVID-19 treatment. Pharmacol Res Perspect, 2022. 10(1): p. e00909.
19. Khiali, S., et al., Comprehensive review on molnupiravir in COVID-19: a novel promising antiviral to combat the pandemic. Future Microbiol, 2022. 17: p. 377-391.
20. Thye, A. Y, et al., Psychological Symptoms in COVID-19 Patients: Insights into Pathophysiology and Risk Factors of Long COVID-19. Biology (Basel), 2022. 11(1).
21. Thallapureddy, K., et al., Long-Term Complications of COVID-19 Infection in Adolescents and Children. Curr Pediatr Rep, 2022: p. 1-7.
22. Mehandru, S. and M. Merad, Pathological sequelae of long-haul COVID. Nat Immunol, 2022. 23(2): p. 194-202.
23. Joshee, S., N. Vatti, and C. Chang, Long-Term Effects of COVID-19. Mayo Clin Proc, 2022. 97(3): p. 579-599.
24. Han, Q., et al., Long-Term Sequelae of COVID-19: A Systematic Review and Meta-Analysis of One-Year Follow-Up Studies on Post-COVID Symptoms. Pathogens, 2022. 11(2).
25. Desai, A. D., et al., Long-term complications of COVID-19. Am J Physiol Cell Physiol, 2022. 322(1): p. C1-C11.
26. Kim, D., et al., The Architecture of SARS-CoV-2 Transcriptome. Cell, 2020. 181(4): p. 914-921 e10.
27. Snijder, E. J., E. Decroly, and J. Ziebuhr, The Nonstructural Proteins Directing Coronavirus RNA Synthesis and Processing. Adv Virus Res, 2016. 96: p. 59-126.
28. Sola, I., et al., Continuous and Discontinuous RNA Synthesis in Coronaviruses. Annu Rev Virol, 2015. 2(1): p. 265-88.
29. Kaplan, G. and V. R. Racaniello, Construction and characterization of poliovirus subgenomic replicons. J Virol, 1988. 62(5): p. 1687-96.
30. Thumfart, J. O. and G. Meyers, Feline calicivirus: recovery of wild-type and recombinant viruses after transfection of cRNA or cDNA constructs. J Virol, 2002. 76(12): p. 6398-407.
31. Liljestrom, P and H. Garoff, A new generation of animal cell expression vectors based on the Semliki Forest virus replicon. Biotechnology (N Y), 1991. 9(12): p. 1356-61.
32. Khromykh, A. A. and E. G. Westaway, Subgenomic replicons of the flavivirus Kunjin: construction and applications. J Virol, 1997. 71(2): p. 1497-505.
33. Lohmann, V, et al., Replication of subgenomic hepatitis C virus RNAs in a hepatoma cell line. Science, 1999. 285(5424): p. 110-3.
34. Behrens, S. E., et al., Characterization of an autonomous subgenomic pestivirus RNA replicon. J Virol, 1998. 72(3): p. 2364-72.
35. Pang, X., M. Zhang, and A. I. Dayton, Development of Dengue virus type 2 replicons capable of prolonged expression in host cells. BMC Microbiol, 2001. 1: p. 18.
36. Shi, P. Y., M. Tilgner, and M. K. Lo, Construction and characterization of subgenomic replicons of New York strain of West Nile virus. Virology, 2002. 296(2): p. 219-33.
37. Hertzig, T., et al., Rapid identification of coronavirus replicase inhibitors using a selectable replicon RNA. J Gen Virol, 2004. 85(Pt 6): p. 1717-1725.
38. Ge, F, et al., Derivation of a novel SARS-coronavirus replicon cell line and its application for anti-SARS drug screening. Virology, 2007. 360(1): p. 150-8.
39. Ge, F, et al., High-throughput assay using a GFP-expressing replicon for SARS-CoV drug discovery. Antiviral Res, 2008. 80(2): p. 107-13.
40. Almazan, F, C. Galan, and L. Enjuanes, The nucleoprotein is required for efficient coronavirus genome replication. J Virol, 2004. 78(22): p. 12683-8.
41. Wang, J. M., L. F. Wang, and Z. L. Shi, Construction of a non-infectious SARS coronavirus replicon for application in drug screening and analysis of viral protein function. Biochem Biophys Res Commun, 2008. 374(1): p. 138-42.
42. Bartenschlager, R., Hepatitis C virus replicons: potential role for drug development. Nat Rev Drug Discov, 2002. 1(11): p. 911-6.
43. Randall, G. and C. M. Rice, Hepatitis C virus cell culture replication systems: their potential use for the development of antiviral therapies. Curr Opin Infect Dis, 2001. 14(6): p. 743-7.
44. Berkhout, B., et al., TAR-independent activation of the HIV-1 LTR: evidence that tat requires specific regions of the promoter. Cell, 1990. 62(4): p. 757-67.
45. Jakobovits, A., A. Rosenthal, and D. J. Capon, Trans-activation of HIV-1 LTR-directed gene expression by tat requires protein kinase C. EMBO J, 1990. 9(4): p. 1165-70.
46. Jeang, K. T, P. R. Shank, and A. Kumar, Transcriptional activation of homologous viral long terminal repeats by the human immunodeficiency virus type 1 or the human T-cell leukemia virus type I tat proteins occurs in the absence of de novo protein synthesis. Proc Natl Acad Sci USA, 1988. 85(21): p. 8291-5.
47. Selby, M. J., et al., Structure, sequence, and position of the stem-loop in tar determine transcriptional elongation by tat through the HIV-1 long terminal repeat. Genes Dev, 1989. 3(4): p. 547-58.
48. Chang, J. J., et al., Transcriptional and epi-transcriptional dynamics of SARS-CoV-2 during cellular infection. Cell Rep, 2021. 35(6): p. 109108.
49. Davidson, A. D., et al., Characterisation of the transcriptome and proteome of SARS-CoV-2 reveals a cell passage induced in-frame deletion of the furin-like cleavage site from the spike glycoprotein. Genome Med, 2020. 12(1): p. 68.
50. V'Kovski, P., et al., Coronavirus biology and replication: implications for SARS-CoV-2. Nat Rev Microbiol, 2021. 19(3): p. 155-170.
51. Wang, D., et al., The SARS-CoV-2 subgenome landscape and its novel regulatory features. Mol Cell, 2021. 81(10): p. 2135-2147 e5.
52. Yang, Y, et al., Characterizing Transcriptional Regulatory Sequences in Coronaviruses and Their Role in Recombination. Mol Biol Evol, 2021. 38(4): p. 1241-1248.
53. Beigel, J. H., et al., Remdesivir for the Treatment of Covid-19-Final Report. N Engl J Med, 2020. 383(19): p. 1813-1826.
54. Gordon, C. J., et al., The antiviral compound remdesivir potently inhibits RNA-dependent RNA polymerase from Middle East respiratory syndrome coronavirus. J Biol Chem, 2020. 295(15): p. 4773-4779.
55. Grein, J., et al., Compassionate Use of Remdesivir for Patients with Severe Covid-19. N Engl J Med, 2020. 382(24): p. 2327-2336.
56. Kokic, G., et al., Mechanism of SARS-CoV-2 polymerase staffing by remdesivir. Nat Commun, 2021. 12(1): p. 279.
57. Wang, Q., et al., Structural Basis for RNA Replication by the SARS-CoV-2 Polymerase. Cell, 2020. 182(2): p. 417-428 e13.
58. Liu, S., et al., Stable Cell Clones Harboring Self-Replicating SARS-CoV-2 RNAs for Drug Screen. J Virol, 2022: p. jvi0221621.
59. Ricardo-Lax, I., et al., Replication and single-cycle delivery of SARS-CoV-2 replicons. Science, 2021. 374(6571): p. 1099-1106.
60. Zhang, Q. Y, et al., SARS-CoV-2 replicon for high-throughput antiviral screening. J Gen Virol, 2021. 102(5).
61. Kotaki, T, et al., A PCR amplicon-based SARS-CoV-2 replicon for antiviral evaluation. Sci Rep, 2021. 11(1): p. 2229.
62. He, X., et al., Generation of SARS-CoV-2 reporter replicon for high-throughput antiviral screening and testing. Proc Natl Acad Sci USA, 2021. 118(15).
63. Zhang, H., et al., Construction and characterization of two SARS-CoV-2 minigenome replicon systems. J Med Virol, 2022.
64. Nguyen, H. T, et al., Construction of a Noninfectious SARS-CoV-2 Replicon for Antiviral-Drug Testing and Gene Function Studies. J Virol, 2021. 95(18): p. e0068721.
65. Wang, B., et al., Construction of Non-infectious SARS-CoV-2 Replicons and Their Application in Drug Evaluation. Virol Sin, 2021. 36(5): p. 890-900.
66. Luo, Y, et al., Engineering a Reliable and Convenient SARS-CoV-2 Replicon System for Analysis of Viral RNA Synthesis and Screening of Antiviral Inhibitors. mBio, 2021. 12(1).
67. Tanaka, T., et al., Establishment of a stable SARS-CoV-2 replicon system for application in high-throughput screening. Antiviral Res, 2022. 199: p. 105268.

1. A construct comprising a SARS-CoV-2 (SARS2) replicon, wherein the construct comprises 5′ to 3′:
a) two or more promoter sequences;
b) a nucleic acid sequence encoding SARS2 non-structural protein 1;
c) a first reporter gene;
d) a nucleic acid sequence encoding SARS2 non-structural proteins 2-16;
e) a second reporter gene; and
f) a nucleic acid sequence encoding SARS2 nucleocapsid protein.

2. The construct of claim 1 further comprising a 5′ untranslated region (UTR) that is 5′ of the nucleic acid sequence encoding SARS2 non-structural protein 1.

3. The construct of claim 2, wherein the 5′ UTR is 5′ of the nucleic acid sequence encoding SARS2 non-structural protein 1, and is 3′ of the two or more promoter sequences.

4. The construct of claim 1 further comprising a 3′ untranslated region (UTR) that is 3′ of the nucleic acid sequence encoding SARS2 nucleocapsid protein.

5. The construct of claim 1, wherein the two or more promoters comprise a eukaryotic promoter and a prokaryotic promoter.

6. The construct of claim 5, wherein the eukaryotic promoter is selected from a HIV-1 long terminal repeat (LTR) promoter, a HIV-2 LTR promoter, a beta actin promoter, a cytomegalovirus (CMV) promoter, an human elongation factor-1 alpha (EF-1 alpha; EF1a) promoter, a phosphoglycerate kinase (PGK1) promoter, a simian virus 40 (SV40) promoter, a polyubiquitin C gene (UBC) promoter, a human T-lymphotropic virus type 1 (HTLV-1) LTR promoter, a human T-lymphotropic virus type 2 (HTLV-2) LTR promoter, a simian immunodeficiency virus (SIV) LTR promoter, a visna virus LTR promoter, a feline immunodeficiency virus (FIV) LTR promoter, an equine infectious anemia virus (EIAV) LTR promoter, a simian T-cell lymphoma virus (STLV) LTR promoter, a bovine leukemia virus (BLV) LTR promoter, a simian foamy virus (SFV) LTR promoter, and a bovine foamy virus (BFV) LTR promoter.

7. The construct of claim 5, wherein the prokaryotic promoter is selected from a T7 promoter, a lac promoter, a Sp6 promoter, a tac promoter, a tet promoter, a trp promoter, a trc promoter, a T3 promoter, and a T5 promoter.

8. The construct of claim 1, wherein the first report gene and the second reporter gene are optically detectable.

9. The construct of claim 1, wherein the first report gene and the second reporter gene are optically distinguishable.

10. A construct comprising a SARS-CoV-2 (SARS2) replicon, wherein the construct comprises 5′ to 3′:
a) an HIV-1 promoter sequence;
b) a T7 promoter sequence;
c) a hammerhead virus ribozyme sequence;
d) a SARS2 5′ UTR sequence;
e) a nucleic acid sequence encoding SARS2 non-structural protein 1;
f) a P2A self-cleaving peptide sequence;
g) a first reporter gene;
h) an internal ribozyme entry site;
j) a nucleic acid sequence encoding SARS2 non-structural proteins 2-16;
k) a second reporter gene;
l) a nucleic acid sequence encoding SARS2 nucleocapsid protein;
m) a 3′ UTR sequence;
n) a hepatitis delta virus ribozyme sequence; and
o) a poly-A tail sequence.

11. The construct of claim 1, wherein the construct comprises the sequence of SEQ ID NO:14.

12. A method for screening a test compound for anti-SARS2 activity, wherein the method comprises:
a) incubating the test compound with a cell line comprising the construct of claim 1; and
b) assaying for the presence of expression of the first reporter gene or expression of the second reporter gene,
wherein a decreased expression of the first reporter gene or a decreased expression of the second reporter gene compared to a control indicates that the test compound comprises anti-SARS2 activity.

13. A method for screening a test compound for anti-SARS2 activity, wherein the method comprises:
a) incubating the test compound with a cell line comprising the construct of claim 10; and
b) assaying for the presence of expression of the first reporter gene or expression of the second reporter gene,
wherein a decreased expression of the first reporter gene or a decreased expression of the second reporter gene compared to a control indicates that the test compound comprises anti-SARS2 activity.

14. A kit for screening a test compound for anti-SARS2 activity, wherein the kit comprises the construct of claim 1.

15. A kit for screening a test compound for anti-SARS2 activity, wherein the kit comprises the construct of claim 10.

16. The kit of claim 14, wherein the kit further comprises:
a) one or more primer sets to detect one or more genes of the construct;
b) one or more cell lines to be transfected with the construct; or
c) reagents for detection and quantitation of the two or more reporter genes of the construct.

17. The construct of claim 10, wherein the construct comprises the sequence of SEQ ID NO:14.

18. The kit of claim 15, wherein the kit further comprises:
a) one or more primer sets to detect one or more genes of the construct;
b) one or more cell lines to be transfected with the construct; or
c) reagents for detection and quantitation of the two or more reporter genes of the construct.
